(19) United States
(12) Patent Application Publication
(10) Pub. No.: US 2026/0033466 A1
(43) Pub. Date: Feb. 5, 2026

(54) METHOD AND ANIMAL MODEL FOR INDUCING BCC TUMORS

(71) Applicant: FELDAN BIO INC., Quebec (CA)

(72) Inventors: Nancy MESSIER, Quebec (CA); Frederic COUTURE, Quebec (CA)

(21) Appl. No.: 19/106,825

(22) PCT Filed: Aug. 30, 2023
(86) PCT No.: PCT/CA2023/051147

§ 371 Date: Feb. 26, 2025

Related U.S. Application Data
(60) Provisional application No. 63/373,956, filed on Aug. 30, 2022.

(51) Int. Cl. A01K 67/0275; A61K 49/00; A61P 35/00; C12N 15/113

(52) U.S. Cl. CPC A01K 67/0275; A61K 49/0008; A61P 35/00; C12N 15/1138; A01K 2217/058; A01K 2217/203; A01K 2217/206; A01K 2227/105; A01K 2267/0331; C12N 2310/531

(57) ABSTRACT

Methods for inducing basal cell carcinoma (BCC) or BCC tumors, as well as an inducible non-human animal models of BCC, are defined herein. The methods and animal models comprise targeting Ptch1 and/or a tumor suppressor gene via conditional expression of one or more short hairpin RNAs (shRNAs) in the skin of said animals.

The present description relates to a non-human animal model of basal cell carcinoma (BCC) or BCC tumors, as well as methods for inducing BCC or BCC tumors. More specifically, the present description related to a non-human animal conditionally and inducibly expressing a shRNA targeting Ptch1.

The present description refers to a number of documents, the contents of which are herein incorporated by reference in their entirety.

### BACKGROUND

Basal cell carcinoma (BCC) is one of the most common types of skin cancer and is often caused by exposure to ultraviolet (UV) light or radiation, and/or certain genetic mutations (such as in the Hedgehog signaling pathway), leading to the appearance of visible tumors in the skin. Several mouse models of BCC exist, including conditional knockouts (e.g., in the skin) of genes of the Hedgehog signaling pathway like Ptch1 (Nitzki et al., 2012). However, these mouse models have several disadvantages. First, Ptch1 knockout in mice is lethal and therefore most models involve a single copy deletion of Ptch1 (heterozygous expression of Ptch1; Ptch1^{neo12+}). Ptch1 expression is therefore reduced but is still significantly maintained. Furthermore, knockouts targeting specific exons of Ptch1 have been shown to be less effective due to the appearance of alternative splicing leading to a functional protein (Nitzki et al., 2012). Second, these models require elevated levels and repeated exposures of UV or ionizing radiation to induce BCC tumors and take a long time to develop these tumors. Third, the tumors observed in these models are often not BCC tumors or tumors typically seen in BCC patients, and often these mice develop secondary pathologies unrelated to BCC. Thus, there remains a need for improved animal models of BCC that closely recapitulates human disease.

### SUMMARY

In a first aspect, described herein is a genetically-modified non-human animal whose genome comprises one or more sequences encoding a short hairpin RNA (shRNA) targeting Ptch1, and a shRNA targeting p53 or other tumor suppressor genes, wherein expression or co-expression of said shRNAs is inducible and in the skin of said non-human animal.

In a further aspect, described herein is a genetically-modified non-human animal whose genome comprises:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
a gene encoding a site-specific recombinase that cleaves the upstream cleavable sequence, wherein said gene is under the control of a skin cell-specific promoter and wherein expression of said gene is inducible.

In a further aspect, described herein is a method of producing an inducible non-human animal, the method comprising:
inserting an inducible cassette into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
inserting into the genome of the non-human animal an inducible gene encoding a site-specific recombinase that cleaves the upstream cleavable sequence, wherein said gene is under the control of a skin cell-specific promoter.

In a further aspect, described herein is a method for inducing basal cell carcinoma (BCC) or BCC tumor(s) in a non-human animal, the method comprising:
providing a non-human animal whose genome comprises:
   an inducible cassette inserted into a genetic safe harbour site, wherein said cassette comprises:
      an upstream cleavable sequence that blocks expression of downstream sequences or genes;
      a first downstream sequence encoding a short hairpin RNA (shRNA) targeting Ptch1; and
      a second downstream sequence encoding a shRNA targeting p53;
   and
   a gene encoding a site-specific recombinase that cleaves the upstream cleavable sequence,
   wherein said gene is under the control of a skin cell-specific promoter and wherein expression of said gene is inducible;
and
administering a compound for inducing expression of the recombinase.

In a further aspect, described herein is a cell comprising:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
a gene encoding a site-specific recombinase that cleaves the upstream cleavable sequence, wherein said gene is under the control of a skin cell-specific promoter and wherein expression of said gene is inducible.

In a further aspect, described herein is a method of evaluating in a non-human animal, a candidate compound or intervention useful in the treatment, reduction or prevention of basal cell carcinoma (BCC) or BCC tumors, the method comprising the steps of:
providing the non-human animal as defined herein or a non-human animal produced by the method as defined herein;
inducing BCC or BCC tumor(s) by the method as defined herein; and
administering the candidate compound or intervention to the non-human animal.

In a further aspect, described herein is a use of the non-human animal as defined herein for evaluating a candidate compound or intervention useful in the treatment, reduction or prevention of basal cell carcinoma (BCC) or BCC tumors.

In a further aspect, described herein is a gene cassette or vector (e.g., plasmid) comprising: an upstream cleavable sequence that blocks expression of downstream sequences or genes;
a first downstream sequence encoding a short hairpin RNA (shRNA) targeting Ptch1; and
a second downstream sequence encoding a shRNA targeting p53.

In a further aspect, described herein is a nucleotide sequence comprising:
a sequence encoding inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
a sequence encoding a gene encoding a site-specific recombinase that cleaves the upstream cleavable sequence, wherein said gene is under the control of a skin cell-specific promoter and wherein expression of said gene is inducible.

In a further aspect, described herein is a short hairpin RNA targeting Ptch1, said shRNA being encoded by a sequence having at least 50, 60, 70, 80, 90, 95, or 99% identity to the sequence of any one of SEQ ID NOs: 1-10.

In a further aspect, described herein is a short hairpin RNA targeting Ptch1, said shRNA being encoded by the sequence of any one of SEQ ID NOs: 1-10.

In a further aspect, described herein is a short hairpin RNA targeting Ptch1, said shRNA targeting a portion or region on SEQ ID NO: 12 that is at least 50, 60, 70, 80, 90, 95, 99%, or 100% identical to one or more of:
(i) Nucleobases 2776-2796 of SEQ ID NO: 12;
(ii) Nucleobases 3601-3621 of SEQ ID NO: 12;
(iii) Nucleobases 442-462 of SEQ ID NO: 12;
(iv) Nucleobases 443-463 of SEQ ID NO: 12;
(v) Nucleobases 2526-2546 of SEQ ID NO: 12;
(vi) Nucleobases 1912-1933 of SEQ ID NO: 12;
(vii) Nucleobases 964-986 of SEQ ID NO: 12;
(viii) Nucleobases 2859-2880 of SEQ ID NO: 12;
(ix) Nucleobases 2864-2884 of SEQ ID NO: 12; and
(x) Nucleobases 824-844 of SEQ ID NO: 12.

In a further aspect, described herein is a composition or kit comprising one or more of the shRNAs as defined herein.

In a further aspect, described herein is a non-human animal expressing the gene cassette or vector as defined herein.

In a further aspect, described herein is a cell comprising or expressing the gene cassette or vector as defined herein.

In a further aspect, described herein is a genetically-modified non-human animal whose genome comprises:
an inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53.

In a further aspect, described herein is a cell comprising:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows a schematic representation of an inducible mouse model of BCC according to one example embodiment.

FIG. 2 shows the results of knockdown efficiency of different shRNAs targeting mouse Ptch1 (shPtch1) in a reporter assay. Knockdown efficiency was calculated by Ptch1 mRNA expression normalized to shRen713 (positive control).

FIG. 3 shows the representative result of immunofluorescence (IF) staining of GFP in the skin of untreated (i.e., uninduced) WT mice (FIG. 3A) or mice that were genetically modified as described in FIG. 1 (i.e., BCC mice) at 9 weeks (FIG. 3B). FIG. 3C shows the representative result of IF of GFP and keratin 14 (K14 or CK14; skin cells) in BCC mice 14 weeks after induction with tamoxifen/doxycycline and UV-B radiation. DAPI levels indicate nuclear staining. Positive GFP fluorescence is indicative of Ptch1 and p53 shRNA expression.

FIG. 4 shows the representative result of IF staining of GFP in the skin of BCC mice, either untreated, or after induction with either tamoxifen alone, doxycycline alone, or tamoxifen/doxycycline, in the absence of UV-B radiation. Positive GFP fluorescence is indicative of Ptch1 and p53 shRNA expression. Nucleus staining done with DAPI (Blue). FIG. 5 shows the representative result of IF staining of Ptch1 or GFP in the skin of BCC mice, 14 weeks after induction with either tamoxifen/doxycycline, UV-B radiation, and/or untreated. DAPI levels indicate nuclear staining. Positive GFP fluorescence is indicative of Ptch1 and p53 shRNA expression.

FIG. 6 shows the quantitative expression of Ptch1 based on the IF results of FIG. 5.

FIG. 7 shows the representative result of hematoxylin and eosin (H&E) staining of the skin of BCC mice upon induction with either tamoxifen/doxycycline or untreated in the presence of UV-B at baseline, 6 weeks, 14 weeks, or 20 weeks after induction.

FIG. 8 shows a representative image of two BCC mice induced with tamoxifen/doxycycline and UV-B. Arrows point to BCC tumors 23 weeks post-induction.

FIG. 9 shows the representative immunohistochemical (IHC) staining of Ki67 (FIG. 9A) and Gli1 (FIG. 9B), and H&E staining (FIG. 9C), on BCC tumors upon induction with tamoxifen/doxycycline and UV-B at 23 weeks of induction.

FIG. 10A shows the representative result of the effect of daily treatment of four mice (4447, 4455, 4449, and 4571) with a Gli-1-targeting topical formulation on the tumor volume of BCC mice induced with tamoxifen/doxycycline and UV-B. FIG. 10B shows images of the tumor volume reduction at different time points of induced mouse (4571) treated with topical formulation. FIG. 10C shows GFP expression of the four treated mice at 28 days after treatment, which is indicative of Ptch1 and p53 shRNA expression.

FIG. 11 shows the representative result of the effect of daily treatment with a Gli-1-targeting topical formulation on the tumors of three BCC mice (4686, 4683 and 4707) induced with tamoxifen/doxycycline and UV-B. Treatment began at 33-34 weeks post-induction. Representative images of treated and non-treated tumors at different time points of induced mice are shown.

FIG. 12 shows the representative result of the effect of daily treatment with oral vismodegib (via gastric probe gavage at 5 mg/kg), on the tumor volume of BCC mice induced with tamoxifen/doxycycline and UV-B. Treatment began at 37-43 weeks post-induction. Representative images of the tumors (circle) at different time points of two different induced mice treated with oral vismodegib are shown.

FIG. 13 shows the representative result of the effect of treatment with intraperitoneal (i.p.) everolimus (3 mg/kg) in BCC mice induced with tamoxifen/doxycycline and UV-B. Treatment began at 37-43 weeks post-induction. Representative images of the tumors (circle) at different time points of two different induced mice treated with i.p. everolimus are shown.

### SEQUENCE LISTING

This application contains a Sequence Listing in computer readable form created Aug. 30, 2023. The computer readable form is incorporated herein by reference.

TABLE 1
Sequence Listing

| SEQ ID NO: | Description |
|---|---|
| 1 | Ptch1.2559 shRNA |
| 2 | Ptch1.3384 shRNA |
| 3 | Ptch1.226 shRNA |
| 4 | Ptch1.225 shRNA |
| 5 | Ptch1.2309 shRNA |
| 6 | Ptch1.1695 shRNA |
| 7 | Ptch1.747 shRNA |
| 8 | Ptch1.2643 shRNA |
| 9 | Ptch1.2647 shRNA |
| 10 | Ptch1.607 shRNA |
| 11 | p53.1224 shRNA |
| 12 | Mouse Ptch1 (NCBI Reference Sequence: NM_008957.3) |

### DETAILED DESCRIPTION

In a first aspect, described herein is a conditional and inducible non-human animal model of basal cell carcinoma (BCC) or BCC tumor induction. Generally, the non-human animal is genetically-engineered to inducibly and conditionally express both a short hairpin RNA (shRNA) targeting Ptch1 and a shRNA targeting p53 tumor suppressor in basal skin cells.

The expression “inducible” as used herein refers to inducing expression of one or more genes, or gene cassette, via a stimulus. The stimulus may be, for example, a compound, protein, or molecule that binds an estrogen receptor (e.g., tamoxifen), antibiotics (e.g., tetracycline, doxycycline), or transcriptional activators (transactivators) (e.g., rtTA, rtTA2). The stimulus may be an external stimulus. For example, the stimulus may be administered directly into the non-human animal for induction of the corresponding gene expression. The stimulus may also be an internal stimulus that is produced internally (i.e., in the non-human animal). The expression of the gene encoding the internal stimulus, or production of the internal stimulus, may be induced by an external stimulus. In some cases, “inducible” refers to an inducible non-human animal model whereby certain symptoms of a disease or disorder are induced upon administration of one or more stimuli.

In some aspects, “non-human animal” may be a mammal and may include but is not limited to any rodent animal (e.g., mouse, rat, hamster, or guinea pig), ferret, rabbit, pig, monkey, or primate.

In some aspects, “shRNA” (short or small hairpin RNA) refers to a short RNA molecule comprising a hairpin turn which silences target gene expression via RNA interference. In some aspects, the shRNA may be at least 30, 35, 40, 45, 50, 55, 60, 65, 70, 75, 80, 85, 90, 95, or 100 nucleotides in length. The structure of shRNAs generally comprises a loop flanked by two stems. The first stem typically comprises the target sequence, and the second stem comprises the reverse complement of said target sequence. As defined herein, the term “target region or sequence” refers to the target sequence on the target gene upon which the shRNA binds.

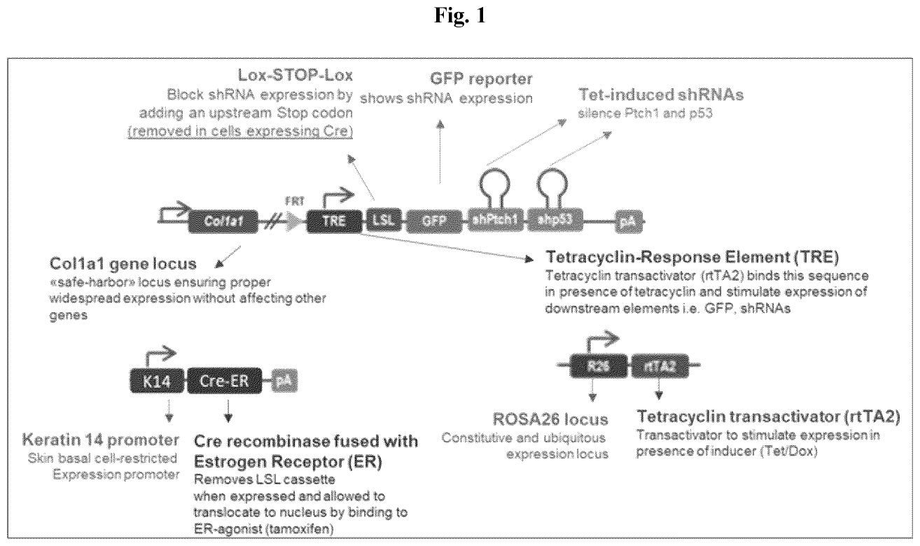

In some aspects, the non-human animal model of BCC or BCC tumor is as depicted in FIG. 1. The inducible cassette as shown includes a sequence encoding for a shRNA targeting Ptch1 gene and a sequence encoding for a shRNA targeting p53 tumor suppressor gene. The cassette also includes a gene encoding for GFP, which acts as a marker for shRNA expression. Upstream of the genes, the cassette includes a Lox-STOP-Lox site (i.e., LoxP site) before a tetracycline-response element (TRE) to prevent any potential leakiness of the TRE. The inducible cassette is inserted into the Col1a1 safe-harbor site.

In some embodiments, described herein is a genetically-modified non-human animal whose genome comprises one or more sequences encoding a short hairpin RNA (shRNA) targeting Ptch1, and a shRNA targeting p53 or other tumor suppressor genes, wherein expression or co-expression of said shRNAs is inducible and in the skin of said non-human animal.

In some embodiments, described herein is a genetically-modified non-human animal whose genome comprises: an inducible cassette inserted into a genetic safe harbour site, wherein said cassette comprises: an upstream cleavable sequence that blocks expression of downstream sequences or genes; a first downstream sequence encoding a short hairpin RNA (shRNA) targeting Ptch1; and a second downstream sequence encoding a shRNA targeting p53; and a gene encoding a site-specific recombinase that cleaves the upstream cleavable sequence, wherein said gene is operably-linked to or is under the control of a skin cell or skin basal cell-specific promoter and wherein expression of said gene is inducible.

In some aspects, the genetic safe harbour (GSH) site may be any known GSH allowing for stable integration of new genetic material ensuring its function predictably and which does not cause alterations of the host genome posing a risk to the host cell or organism. GSHs may include but are not limited to Col1a1, ROSA26, AAVS1, or CCR5.

In some aspects, the upstream cleavable sequence is or comprises a loxP site (i.e., Lox-STOP-Lox site). The upstream cleavable sequence may also include a flippase recognition target (FRT) site in place or in addition to the loxP site. The presence of the upstream cleavable sequence inhibits expression/transcription of downstream sequences. However, when said upstream cleavable sequence is cleaved, expression/transcription of downstream sequences is permitted. The upstream cleavable sequence may be specific for a particular recombinase. For example, the Cre recombines is specific for the loxP site, and expression/transcription of downstream sequences is permitted upon recombination of the loxP site from the cassette by Cre recombinase. Similarly, the flippase recombinase is specific for the FRT site.

In some aspects, the site-specific recombinase is fused to a receptor such that when a ligand or stimulus binds to said receptor, the recombinase is activated (e.g., activation of enzymatic function or translocation to the substrate location). In some aspects, binding of the ligand or stimulus to the receptor induces translocation of the recombinase into the nucleus and subsequent substrate (i.e., upstream cleavable sequence) site. For example, the recombinase Cre may be fused to an estrogen receptor (e.g., ERT2) (i.e., Cre-ER), whereby binding of tamoxifen to Cre-ER induces translocation of the Cre-ER to the nucleus and subsequent recombination of loxP. In some aspects, tamoxifen is administered to the non-human animal for the activation of Cre. In some aspects, the gene encoding the recombinase is operably-linked to or is under the control of a skin cell or skin basal cell-specific promoter. Therefore, the Cre recombinase may only be expressed in skin cells or skin basal cells. Examples of skin cell or skin basal cell-specific promoters may include but are not limited to keratin 14 (K14), Krt6a, or K5.

In some aspects, the cassette defined herein further comprises an inducible promoter operably-linked to or which controls the expression of the upstream cleavable sequence, first and second downstream sequences, wherein said inducible promoter is regulated by a transcriptional activator protein. The inducible promoter may be or comprise a tetracycline-response element (TRE).

The transcriptional activator protein may be administered directly into the non-human animal to activate the inducible promoter and expression/transcription of the downstream sequences. In some cases, the genome of the non-human animal further comprises a gene encoding the transcriptional activator protein. Said gene encoding the transcriptional activator protein may be inserted into a constitutively expressed gene locus (e.g., ROSA26). In some cases, expression of the gene encoding the transcriptional activator protein is inducible. For example, the transcriptional activator may be a tetracycline transactivator (e.g., rtTA or rtTA2) and expression of the gene encoding tetracycline transactivator may be induced/activated in the presence or administration of tetracycline and/or doxycycline.

In some embodiments, the cassette further comprises a third downstream gene encoding a tag or marker, such as fluorescent marker or protein, which when expressed, confirms expression of the one or more sequences (e.g., shRNAs) in the cassette. The marker may include but is not limited to green fluorescent protein [GFP]. Any marker or tag may conceivably be used.

In some embodiments, the shRNA targeting mouse Ptch1 may be specific to any corresponding portion or target region of Ptch1 (SEQ ID NO: 12). In some cases, the non-human animal defined herein expresses at least one of the shRNAs comprising or encoded by SEQ ID NOs: 1-10. In some cases, the non-human animal defined herein expresses 1, 2, 3, 4, 5, 6, 7, 8, 9, or 10 of the shRNAs comprising or encoded by SEQ ID NOs: 1-10. In some cases, the non-human animal defined herein expresses at least one of the shRNAs comprising or encoded by SEQ ID NOs: 1-10 in combination with other Ptch1-targeting shRNAs. In some cases, the sequence encoding the shRNA targeting mouse Ptch1 comprises or is encoded by a sequence that is at least 50, 60, 70, 80, 90, 95, or 99% identical to the sequence of any one of SEQ ID NOs: 1-10. In some cases, the sequence encoding the shRNA targeting Ptch1 comprises or is encoded by the sequence of any one of SEQ ID NOs: 1-10. In some aspects, the shRNA targeting Ptch1 comprises or is encoded by a sequence that is at least 50, 60, 70, 80, 90, 95, 99, or 100% complementary or corresponding to a portion (e.g., equal-length portion) of a target region in Ptch1. The target region of the shRNA of SEQ ID NOs: 1-10 may comprise the portions of mouse Ptch1 (SEQ ID NO: 12) as described in Table 2.

TABLE 2
Position of shRNA Target Region
in Mouse Ptch1 (SEQ ID NO: 12)

| | Position of shRNA Target |
|---|---|
| | Region in Mouse Ptch1 |
| Ptch1 shRNA (SEQ ID NO) | (SEQ ID NO: 12) |
| Ptch1.2559 shRNA (SEQ ID NO: 1) | Nucleobases 2776-2796 |
| Ptch1.3384 shRNA (SEQ ID NO: 2) | Nucleobases 3601-3621 |
| Ptch1.226 shRNA (SEQ ID NO: 3) | Nucleobases 442-462 |
| Ptch1.225 shRNA (SEQ ID NO: 4) | Nucleobases 443-463 |
| Ptch1.2309 shRNA (SEQ ID NO: 5) | Nucleobases 2526-2546 |
| Ptch1.1695 shRNA (SEQ ID NO: 6) | Nucleobases 1912-1933 |
| Ptch1.747 shRNA (SEQ ID NO: 7) | Nucleobases 964-986 |
| Ptch1.2643 shRNA (SEQ ID NO: 8) | Nucleobases 2859-2880 |
| Ptch1.2647 shRNA (SEQ ID NO: 9) | Nucleobases 2864-2884 |
| Ptch1.607 shRNA (SEQ ID NO: 10) | Nucleobases 824-844 |

In some embodiments, described herein is a shRNA targeting Ptch1 comprising or encoded by a sequence that is at least 50, 60, 70, 80, 90, 95, or 99% identical to the sequence of any one of SEQ ID NOs: 1-10. In some cases, the sequence encoding the shRNA targeting Ptch1 comprises or is encoded by the sequence of any one of SEQ ID NOs: 1-10. In some aspects, the shRNA targeting Ptch1 comprises or is encoded by a sequence that is at least 50, 60, 70, 80, 90, 95, 99, or 100% complementary or corresponding to a portion (e.g., equal-length portion) of a target region in Ptch1 or to one or more of the target regions described in Table 2.

In some embodiments, described herein is a composition comprising one or more shRNAs targeting Ptch1 comprising or encoded by a sequence that is at least 50, 60, 70, 80, 90, 95, or 99% identical to the sequence of any one of SEQ ID NOs: 1-10. In some cases, the composition one or more of the shRNA targeting Ptch1 comprising or encoded by SEQ ID NOs: 1-10. In some aspects, the composition comprises a shRNA targeting Ptch1 comprising or encoded by a sequence that is at least 50, 60, 70, 80, 90, 95, 99, or 100% complementary or corresponding to a portion (e.g., equal-length portion) of a target region in Ptch1 or to one or more of the target regions described in Table 2. In some aspects, the composition further comprises a pharmaceutically acceptable carrier, diluent, or excipient.

In some embodiments, the shRNA targeting p53 may be specific to any portion or target region of p53. In some cases, the sequence encoding the shRNA targeting p53 comprises or is encoded by a sequence that is at least 50, 60, 70, 80, 90, 95, or 99% identical to the sequence of SEQ ID NO:11. In some cases, the sequence encoding the shRNA targeting p53 comprises or is encoded by the sequence of SEQ ID NO: 11. In some aspects, the shRNA targeting p53 comprises or is encoded by a sequence that is at least 50, 60, 70, 80, 90, 95, 99, or 100% complementary or corresponding to a portion (e.g., equal-length portion) of a target region in p53.

In some embodiments, other tumor suppressor genes, other than p53, may be targeted via shRNA in combination with shRNA targeting Ptch1 in the non-human animal defined herein. For example, MicroRNA-451a, MicroRNA-203, PTPN14 or members of the RAS proto-oncogene family may be targeted and silenced to induce BCC.

In some aspects, described herein is a method of producing a inducible non-human animal model of basal cell carcinoma (BCC). The method may comprise inserting an inducible cassette into a genetic safe harbour site, wherein said cassette comprises an upstream cleavable sequence that blocks expression of downstream sequences or genes; a first downstream sequence encoding a short hairpin RNA (shRNA) targeting Ptch1; and a second downstream sequence encoding a shRNA targeting p53; and inserting into the genome of the non-human animal an inducible gene encoding a site-specific recombinase that cleaves the upstream cleavable sequence, wherein said gene is operably-linked to or under the control of a skin cell or skin basal cell-specific promoter. In some aspects, the method defined herein is for producing the non-human animal as defined herein.

In some aspects, described herein is a method for inducing basal cell carcinoma (BCC) or BCC tumor(s) in a non-human animal. The method may comprise: providing a non-human animal model whose genome comprises: an inducible cassette inserted into a genetic safe harbour site, wherein said cassette comprises: an upstream cleavable sequence that blocks expression of downstream sequences or genes; a first downstream sequence encoding a short hairpin RNA (shRNA) targeting Ptch1; and a second downstream sequence encoding a shRNA targeting p53; and a gene encoding a site-specific recombinase that cleaves the upstream cleavable sequence, wherein said gene is operably-linked to or under the control of a skin cell or skin basal cell-specific promoter and wherein expression of said gene is inducible; and administering a compound for inducing expression of the recombinase (e.g., tamoxifen). In some aspects, the method further comprises administering a compound for inducing expression of the transcriptional activator protein (e.g., tetracycline and/or doxycycline). In some aspects, the method further comprises exposing the non-human animal to ultraviolet radiation (e.g., UV-B) and/or ionizing radiation. The radiation may be given at any dosage. For example, the radiation dosage may be at least 50, 100, 150, 200, 250, 300, 400, or 500 mJ/cm². In some aspects, the method defined herein is for inducing basal cell carcinoma (BCC) or BCC tumor(s) in the non-human animal as defined herein.

In some aspects, described herein is an isolated cell from the non-human animal as defined in herein or from a non-human animal produced by the method defined herein.

In some aspects, described herein is a cell comprising: an inducible cassette inserted into a genetic safe harbour site, wherein said cassette comprises: an upstream cleavable sequence that blocks expression of downstream sequences or genes; a first downstream sequence encoding a short hairpin RNA (shRNA) targeting Ptch1; and a second downstream sequence encoding a shRNA targeting p53; and a gene encoding a site-specific recombinase that cleaves the upstream cleavable sequence, wherein said gene is operably-linked to or under the control of a skin cell or skin basal cell-specific promoter and wherein expression of said gene is inducible. In some aspects, said cell is a skin cell, skin basal cell, germ cell, stem cell, embryonic stem cell, multipotent stem cell, pluripotent stem cell, totipotent stem cells, or a fertilized cell (e.g., oocyte, zygote).

In some aspects, described herein is a method of evaluating in a non-human animal, a candidate compound or intervention useful in the treatment, reduction or prevention of basal cell carcinoma (BCC) or BCC tumors. The method may comprise the steps of: providing the non-human animal model as defined herein or a non-human animal produced by the method defined herein; inducing BCC or BCC tumor(s) by the method defined herein; and administering the candidate compound or intervention to the non-human animal.

In some aspects, described herein is a gene cassette or plasmid comprising: an upstream cleavable sequence that blocks expression of downstream sequences or genes; a first downstream sequence encoding a short hairpin RNA (shRNA) targeting Ptch1; and a second downstream sequence encoding a shRNA targeting p53. In some aspects, the expression of the cassette or plasmid is inducible.

In some aspects, described herein is a nucleotide sequence comprising: a sequence encoding inducible cassette inserted into a genetic safe harbour site, wherein said cassette comprises: an upstream cleavable sequence that blocks expression of downstream sequences or genes; a first downstream sequence encoding a short hairpin RNA (shRNA) targeting Ptch1; and a second downstream sequence encoding a shRNA targeting p53; and a sequence encoding a gene encoding a site-specific recombinase that cleaves the upstream cleavable sequence, wherein said gene is operably-linked to or under the control of a skin cell or skin basal cell-specific promoter and wherein expression of said gene is inducible.

In some aspects, described herein is a use of the non-human animal defined herein as a non-human animal model of inducible basal cell carcinoma (BCC) or BCC tumors.

In some aspects, described herein is a use of the non-human animal as defined herein for evaluating a candidate compound or intervention useful in the treatment, reduction or prevention of basal cell carcinoma (BCC) or BCC tumors.

### Items

1. A genetically-modified non-human animal whose genome comprises one or more sequences encoding a short hairpin RNA (shRNA) targeting Ptch1, and a shRNA targeting p53 or other tumor suppressor genes, wherein expression or co-expression of said shRNAs is inducible and in the skin of said non-human animal.

2. A genetically-modified non-human animal whose genome comprises:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
a gene encoding a site-specific recombinase that cleaves the upstream cleavable sequence, wherein said gene is under the control of a skin cell-specific promoter and wherein expression of said gene is inducible.

3. The non-human animal of item 2, wherein the cassette comprises an inducible promoter which controls the expression of the upstream cleavable sequence, first and second downstream sequences, wherein said inducible promoter is regulated by a transcriptional activator protein.

4. The non-human animal of item 3, wherein the genome further comprises a gene encoding the transcriptional activator protein, wherein said gene is inserted into a constitutively expressed gene locus (e.g., ROSA26).

5. The non-human animal of item 4, wherein expression of the gene encoding the transcriptional activator protein is inducible.

6. The non-human animal of any one of items 3 to 5, wherein said inducible promoter is or comprises a tetracycline-response element (TRE) and wherein said transcriptional activator is a tetracycline transactivator (e.g., rtTA or rtTA2).

7. The non-human animal of any one of items 3 to 6, wherein expression of the transcriptional activator is induced in the presence of tetracycline and/or doxycycline.

8. The non-human animal of any one of items 2 to 7, wherein said skin cell-specific promoter is or comprises keratin 14 (K14), Krt6a, or K5.

9. The non-human animal of any one of items 2 to 8, wherein said upstream cleavable sequence is or comprises a loxP site (i.e., Lox-STOP-Lox site).

10. The non-human animal of any one of items 2 to 9, wherein said upstream cleavable sequence is or comprises a flippase recognition target (FRT) site.

11. The non-human animal of any one of items 2 to 10, wherein said recombinase is or comprises a Cre recombinase or a flippase recombinase.

12. The non-human animal of any one of items 2 to 11, wherein said recombinase is fused to a receptor, such as an estrogen receptor (e.g., ERT2).

13. The non-human animal of any one of items 2 to 12, wherein activation of the recombinase is induced in the presence of tamoxifen.

14. The non-human animal of any one of items 2 to 13, wherein said cassette further comprises a third downstream gene encoding a fluorescent marker or protein (e.g., green fluorescent protein [GFP]).

15. The non-human animal of any one of items 2 to 14, wherein said genetic safe-harbour site is or comprises the Col1a1 gene locus.

16. The non-human animal of any one of items 2 to 15, wherein the sequence encoding the short hairpin RNA (shRNA) targeting Ptch1 comprises the sequence of any one of SEQ ID NOs: 1-10.

17. The non-human animal of any one of items 2 to 16, wherein the sequence encoding the short hairpin RNA (shRNA) targeting p53 comprises the sequence of SEQ ID NO: 11.

18. The non-human animal of any one of items 2 to 17, wherein said non-human animal is a rodent (e.g., mouse, rat, hamster, or guinea pig), ferret, rabbit, pig, monkey, or primate.

19. The non-human animal of any one of items 2 to 18, for use as a non-human animal model of inducible basal cell carcinoma (BCC).

20. A method of producing an inducible non-human animal, the method comprising:
inserting an inducible cassette into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
inserting into the genome of the non-human animal an inducible gene encoding a site-specific recombinase that cleaves the upstream cleavable sequence, wherein said gene is under the control of a skin cell-specific promoter.

21. The method of item 20, wherein the cassette further comprises an inducible promoter which controls the expression of the upstream cleavable sequence, first and second downstream sequences, wherein said inducible promoter is regulated by a transcriptional activator protein.

22. The method of item 21, further comprising inserting into the genome of the non-human animal a gene encoding the transcriptional activator protein, wherein said gene is inserted into a constitutively expressed gene locus (e.g., ROSA26).

23. The method of item 22, wherein expression of the gene encoding the transcriptional activator protein is inducible.

24. The method of any one of items 21 to 23, wherein said inducible promoter is or comprises a tetracycline-response element (TRE) and wherein said transcriptional activator is a tetracycline transactivator (e.g., rtTA or rtTA2).

25. The non-human animal of any one of items 21 to 24, wherein expression of the transcriptional activator is induced in the presence of tetracycline and/or doxycycline.

26. The method of any one of items 20 to 25, wherein said skin cell-specific promoter is or comprises keratin 14 (K14), Krt6a, or K5.

27. The method of any one of items 20 to 26, wherein said upstream cleavable sequence is or comprises a loxP site (i.e., Lox-STOP-Lox site).

28. The method of any one of items 20 to 27, wherein said upstream cleavable sequence is or comprises a flippase recognition target (FRT) site.

29. The method of any one of items 20 to 28, wherein said recombinase is or comprises a Cre recombinase or a flippase recombinase.

30. The method of any one of items 20 to 29, wherein said recombinase is fused to a receptor, such as an estrogen receptor (e.g., ERT2).

31. The method of any one of items 20 to 30, wherein activation of the recombinase is induced in the presence of tamoxifen.

32. The method of any one of items 20 to 31, wherein said cassette further comprises a third downstream gene encoding a fluorescent marker or protein (e.g., green fluorescent protein [GFP]).

33. The method of any one of items 20 to 32, wherein said genetic safe-harbour site is or comprises the Col1a1 gene locus.

34. The method of any one of items 20 to 33, wherein the sequence encoding the short hairpin RNA (shRNA) targeting Ptch1 comprises the sequence of any one of SEQ ID NOs: 1-10.

35. The method of any one of items 20 to 34, wherein the sequence encoding the short hairpin RNA (shRNA) targeting p53 comprises the sequence of SEQ ID NO: 11.

36. The method of any one of items 20 to 35, wherein said non-human animal is a rodent (e.g., mouse, rat, hamster, or guinea pig), ferret, rabbit, pig, monkey, or primate.

37. The method of any one of items 20 to 36, wherein said non-human animal is the non-human animal as defined in any one of items 1 to 19.

38. A method for inducing basal cell carcinoma (BCC) or BCC tumor(s) in a non-human animal, the method comprising:
providing a non-human animal whose genome comprises:
   an inducible cassette inserted into a genetic safe harbour site, wherein said cassette comprises:
      an upstream cleavable sequence that blocks expression of downstream sequences or genes;
      a first downstream sequence encoding a short hairpin RNA (shRNA) targeting Ptch1; and
      a second downstream sequence encoding a shRNA targeting p53;
   and
   a gene encoding a site-specific recombinase that cleaves the upstream cleavable sequence,
   wherein said gene is under the control of a skin cell-specific promoter and wherein expression of said gene is inducible;
and
administering a compound for inducing expression of the recombinase.

39. The method of item 38, wherein the cassette further comprises an inducible promoter which controls the expression of the upstream cleavable sequence, first and second downstream sequences, wherein said inducible promoter is regulated by a transcriptional activator protein

40. The method of item 39, wherein the genome of the non-human animal further comprises a gene encoding the transcriptional activator protein, wherein said gene is inserted into a constitutively expressed gene locus (e.g., ROSA26).

41. The method of item 40, wherein expression of the gene encoding the transcriptional activator protein is inducible.

42. The method of any one of items 39 to 41, wherein said inducible promoter is or comprises a tetracycline-response element (TRE) and wherein said transcriptional activator is a tetracycline transactivator (e.g., rtTA or rtTA2).

43. The method of any one of items 39 to 42, further comprising administering a compound for inducing expression of the transcriptional activator protein.

44. The method of item 43, wherein the compound for inducing expression of the transcriptional regulator protein is or comprises tetracycline or doxycycline.

45. The method of any one of items 38 to 44, wherein the compound for inducing activation of the recombinase is or comprises tamoxifen.

46. The method of any one of items 38 to 45, further comprising exposing the non-human animal to ultraviolet radiation (e.g., UV-B) and/or ionizing radiation.

47. The method of any one of items 38 to 46, wherein the non-human animal is produced by the method of any one of items 20 to 37.

48. The method of any one of items 38 to 47, wherein the non-human animal is the non-human animal as defined in any one of items 1 to 19.

49. An isolated cell from the non-human animal as defined in any one of items 1 to 19 or from a non-human animal produced by the method of any one of items 20 to 37.

50. A cell comprising:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
a gene encoding a site-specific recombinase that cleaves the upstream cleavable sequence, wherein said gene is under the control of a skin cell-specific promoter and wherein expression of said gene is inducible.

51. The cell of item 50, wherein the cassette comprises an inducible promoter which controls the expression of the upstream cleavable sequence, first and second downstream sequences, wherein said inducible promoter is regulated by a transcriptional activator protein.

52. The cell of item 51, further comprising a gene encoding the transcriptional activator protein, wherein said gene is inserted into a constitutively expressed gene locus (e.g., ROSA26).

53. The cell of item 52, wherein expression of the gene encoding the transcriptional activator protein is inducible.

54. The cell of any one of items 51 to 53, wherein said inducible promoter is or comprises a tetracycline-response element (TRE) and wherein said transcriptional activator is a tetracycline transactivator (e.g., rtTA or rtTA2).

55. The cell of any one of items 51 to 54, wherein expression of the transcriptional activator is induced in the presence of tetracycline and/or doxycycline.

56. The cell of any one of items 50 to 55, wherein said skin cell-specific promoter is or comprises keratin 14 (K14), Krt6a, or K5.

57. The cell of any one of items 50 to 56, wherein said upstream cleavable sequence is or comprises a loxP site (i.e., Lox-STOP-Lox site).

58. The cell of any one of items 50 to 57, wherein said upstream cleavable sequence is or comprises a flippase recognition target (FRT) site.

59. The cell of any one of items 50 to 58, wherein said recombinase is or comprises a Cre recombinase or a flippase recombinase.

60. The cell of any one of items 50 to 59, wherein said recombinase is fused to a receptor, such as an estrogen receptor (e.g., ERT2).

61. The cell of any one of items 50 to 60, wherein activation of the recombinase is induced in the presence of tamoxifen.

62. The cell of any one of items 50 to 61, wherein said cassette further comprises a third downstream gene encoding a fluorescent marker or protein (e.g., green fluorescent protein [GFP]).

63. The cell of any one of items 50 to 62, wherein said genetic safe-harbour site is or comprises the Col1a1 gene locus.

64. The cell of any one of items 50 to 63, wherein the sequence encoding the short hairpin RNA (shRNA) targeting Ptch1 comprises the sequence of any one of SEQ ID NOs: 1-10.

65. The cell of any one of items 50 to 64, wherein the sequence encoding the short hairpin RNA (shRNA) targeting p53 comprises the sequence of SEQ ID NO: 11.

66. The cell of any one of items 50 to 65, wherein said cell is a mammalian cell.

67. The cell of any one of items 50 to 66, wherein said cell is from a non-human animal, such as a rodent (e.g., mouse, rat, hamster, or guinea pig), ferret, rabbit, pig, monkey, or primate.

68. The cell of any one of items 50 to 67, wherein said cell is a skin cell, skin basal cell, germ cell, stem cell, embryonic stem cell, multipotent stem cell, pluripotent stem cell, totipotent stem cells, or a fertilized cell (e.g., oocyte, zygote).

69. A method of evaluating in a non-human animal, a candidate compound or intervention useful in the treatment, reduction or prevention of basal cell carcinoma (BCC) or BCC tumors, the method comprising the steps of:
providing the non-human animal as defined in any one of items 1 to 19 or a non-human animal produced by the method of any one of items 20 to 37;
inducing BCC or BCC tumor(s) by the method of any one of items 38 to 48; and administering the candidate compound or intervention to the non-human animal.

70. Use of the non-human animal as defined in any one of items 1 to 19 for evaluating a candidate compound or intervention useful in the treatment, reduction or prevention of basal cell carcinoma (BCC) or BCC tumors.

71. A gene cassette or vector (e.g., plasmid) comprising:
an upstream cleavable sequence that blocks expression of downstream sequences or genes;
   a first downstream sequence encoding a short hairpin RNA (shRNA) targeting Ptch1; and
a second downstream sequence encoding a shRNA targeting p53.

72. The gene cassette or plasmid of item 71, wherein the expression of the cassette or vector is inducible.

73. A nucleotide sequence comprising:
a sequence encoding inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53;
and
a sequence encoding a gene encoding a site-specific recombinase that cleaves the upstream cleavable sequence, wherein said gene is under the control of a skin cell-specific promoter and wherein expression of said gene is inducible.

74. The nucleotide sequence of item 73, wherein the cassette comprises a sequence encoding an inducible promoter which controls the expression of the upstream cleavable sequence, first and second downstream sequences, wherein said inducible promoter is regulated by a transcriptional activator protein.

75. The nucleotide sequence of item 74, wherein the nucleotide sequence further comprises a sequence encoding a gene encoding the transcriptional activator protein, wherein said gene is inserted into a constitutively expressed gene locus (e.g., ROSA26).

76. The nucleotide sequence of item 75, wherein expression of the gene encoding the transcriptional activator protein is inducible.

77. The nucleotide sequence of any one of items 74 to 76, wherein said inducible promoter is or comprises a tetracycline-response element (TRE) and wherein said transcriptional activator is a tetracycline transactivator (e.g., rtTA or rtTA2).

78. The nucleotide sequence of any one of items 74 to 77, wherein expression of the transcriptional activator is induced in the presence of tetracycline and/or doxycycline.

79. The nucleotide sequence of any one of items 73 to 78, wherein said skin cell-specific promoter is or comprises keratin 14 (K14), Krt6a, or K5.

80. The nucleotide sequence of any one of items 73 to 79, wherein said upstream cleavable sequence is or comprises a loxP site (i.e., Lox-STOP-Lox site).

81. The nucleotide sequence of any one of items 73 to 80, wherein said upstream cleavable sequence is or comprises a flippase recognition target (FRT) site.

82. The nucleotide sequence of any one of items 73 to 81, wherein said recombinase is or comprises a Cre recombinase or a flippase recombinase.

83. The nucleotide sequence of any one of items 73 to 82, wherein said recombinase is fused to a receptor, such as an estrogen receptor (e.g., ERT2).

84. The nucleotide sequence of any one of items 73 to 83, wherein activation of the recombinase is induced in the presence of tamoxifen.

85. The nucleotide sequence of any one of items 73 to 84, wherein said cassette further comprises a third downstream sequence encoding a gene encoding a fluorescent marker or protein (e.g., green fluorescent protein [GFP]).

86. The nucleotide sequence of any one of items 73 to 85, wherein said genetic safe-harbour site is or comprises the Col1a1 gene locus.

87. The nucleotide sequence of any one of items 73 to 86, wherein the sequence encoding the short hairpin RNA (shRNA) targeting Ptch1 comprises the sequence of any one of SEQ ID NOs: 1-10.

88. The nucleotide sequence of any one of items 73 to 87, wherein the sequence encoding the short hairpin RNA (shRNA) targeting p53 comprises the sequence of SEQ ID NO: 11.

89. A short hairpin RNA targeting Ptch1, said shRNA being encoded by a sequence having at least 50, 60, 70, 80, 90, 95, or 99% identity to the sequence of any one of SEQ ID NOs: 1-10.

90. A short hairpin RNA targeting Ptch1, said shRNA being encoded by the sequence of any one of SEQ ID NOs: 1-10.

91. A short hairpin RNA targeting Ptch1, said shRNA targeting a portion or region on SEQ ID NO: 12 that is at least 50, 60, 70, 80, 90, 95, 99%, or 100% identical to one or more of:
(i) Nucleobases 2776-2796 of SEQ ID NO: 12;
(ii) Nucleobases 3601-3621 of SEQ ID NO: 12;
(iii) Nucleobases 442-462 of SEQ ID NO: 12;
(iv) Nucleobases 443-463 of SEQ ID NO: 12;
(v) Nucleobases 2526-2546 of SEQ ID NO: 12;
(vi) Nucleobases 1912-1933 of SEQ ID NO: 12;
(vii) Nucleobases 964-986 of SEQ ID NO: 12;
(viii) Nucleobases 2859-2880 of SEQ ID NO: 12;
(ix) Nucleobases 2864-2884 of SEQ ID NO: 12; and
(x) Nucleobases 824-844 of SEQ ID NO: 12.

92. A composition or kit comprising one or more of the shRNAs as defined in any one of items 89 to 91.

93. The shRNA of any one of items 89-91 or the composition or kit of items 92 for use in inducing BCC in a non-human animal.

94. A non-human animal expressing the gene cassette or vector of item 71 or 72.

95. A cell comprising or expressing the gene cassette or vector of item 71 or 72.

96. A genetically-modified non-human animal whose genome comprises:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53.

97. The genetically modified non-human animal of item 96, wherein the inducible cassette is heterzogyously expressed.

98. The genetically modified non-human animal of item 97, wherein the inducible cassette is homozygously expressed.

99. A cell comprising:
an inducible cassette inserted into a genetic safe harbour site, wherein said cassette comprises:
   an upstream cleavable sequence that blocks expression of downstream sequences or genes;
   a first downstream sequence encoding a short hairpin RNA (shRNA) targeting Ptch1; and
   a second downstream sequence encoding a shRNA targeting p53.

### EXAMPLES

### Example 1: Materials and Methods

### 1.1 Generation of Mice

F1 transgenic animals were generated minimally heterozygous (He) for the shRNA-Ptch1+p53 (shRNA) and rtTA2 (tetracycline-responsive transactivator) loci. The He mice were crossed after acclimatization to generate the F2 to obtain a F2 homozygous (Ho) for the two loci. Female F2 mice were then crossed with male K14-Cre-ER (Jackson Laboratory; Maine, USA) to generated functional triple He mice. Presence of each transgene (shRNA and rtTA2) were verified by PCR. For shRNA, homozygosity was confirmed by breeding statistics when crossed with wild type mice.

### 1.2 Induction of BCC

Triple He mice (6-8 weeks old) were given tamoxifen at 75 mg/kg daily intraperitoneally (75 μL of a 20 mg/mL solution (in corn oil)) for 5-7 days followed by administration of doxycycline via food (Harlan Teklad; 625 mg/kg chow; ad libidum) for 7-14 days. The mice are then UV irradiated at 240 mJ/cm² 3×/week in the UV irradiator (on average 4-5 mins each session). Mice were kept on the diet containing doxycycline for the duration of the study. Mice (6-8 weeks old) were irradiated under anesthesia for three times a week for up to 6 months and their heads were shielded via an aluminum shield. Mice were shaved, depilated, and biopsied (diameter of 5 mm) at different time points and were evaluated consistently for development of microscopic and macroscopic tumors. Mice received a dose of buprenorphine SR (slow release) at the time of the biopsy.

### 1.3 Organ Processing and Histology

Biopsied skin specimens were placed in PFA 4% overnight at 4° C. before being placed in 30% sucrose solution for at least 24 h at 4° C. All tissues were then included in optimal cutting temperature (OCT) compounds (20% sucrose: OCT, 1:1) within 7 days and stored at −80° C. until being sectioned using a cryostat.

Tissues were sectioned as 7 μm sections at 4-5 levels (each spaced by 300 μm) in the sample and placed on a single glass slide. The slides were stored at −80° C. until prepared. For H&E, tissue sections were stained using a standard H&E protocol and then mounted for microscopy. Slides were imaged in an automated slide scanner (PANNORAMIC MIDI II™, 3DHistech™ Ltd.).

For histological staining by immunofluorescence, sections were incubated 5 minutes in room temperature PBS to remove the OCT compound and then drained as much as possible. Slides were then blocked in BSA 3% and stained with anti-GFP antibody (ab6556 Abcam) or anti-Ptch1 (ThermoFisher PA5-87508) or anti-CK14 (BioLegend™ #906004) O/N at 4° C. Sections were then washed thrice (5 minutes each) with PBST and incubated with fluorophore-conjugated antibodies. Following washes (3×5 minutes), 100 μL per slide of ProLong™ Glass NucBlue™ (Invitrogen™) were applied and a coverslip. Slides were incubated overnight in the dark prior to imaging. Slides were imaged within 1-4 days after mounting and left in the dark until that time.

### 1.4 Immunohistochemistry

Tissue sections were incubated in citrate buffer (10 mM sodium citrate pH 6.0) to remove OCT compound. Sections were then placed in prewarmed citrate buffer in a presto and autoclaved for 30 minutes. Upon releasing the pressure, buffer was cooled down by placing the presto on ice. Sections were then washed in Tris buffer saline containing 0.1% Tween-20 (TBST). Sections were then incubated for 15 minutes at room temperature in 3% H₂O₂ to quench endogenous peroxidase activity and then washed thrice in TBST for 5 minutes per wash. Using a Pap Pen™ (Dako), tissue sections were circled with hydrophobic ink to retain liquids on the tissue for further incubations. Slides were then blocked in blocking buffer (TBST with 3% BSA and 0.3% Triton™ X-100) for 30 minutes at room temperature. Slide were then incubated with the antibodies diluted in TBST 3% BSA overnight at 4° C. Antibodies used were Gli1 (Novus NBP1-78259)) diluted 1/50 and Ki67 antibody (ab15580, Abcam) diluted 1/250. Sections were washed thrice in TBST (5 minutes each) and incubated in HRP-conjugated secondary anti-rabbit antibody (1/2000; Jackson ImmunoResearch) for 1 h at room temperature. Sections were again washed thrice and incubated for 1 minutes 30 seconds with SignalStain® DAB (Cell Signaling Technology). Chromogenic reactions were stopped by washing with distilled water. Slides were then counterstained in hematoxylin for 30 seconds and differentiated in NH₄OH 10% for 5 seconds. Slides were then dehydrated by consecutive incubation for 3 minutes each in EtOH 95%, 100%, 100%, xylene and then in xylene again until being mounted in Permount™ solution.

### 1.5 IHC and Immunofluorescence Quantification

Quantification of histological images were performed using the Cell-Quant™ module from the CaseViewer™ software (3DHistech). Immunohistochemistry results can be further evaluated by a semiquantitative approach used to assign an H-score (or “histo” score) to the tissue area of interest. First, membrane staining intensity (0, 1+, 2+, or 3+) is determined for each cell in a fixed field. The H-score may simply be based on a predominant staining intensity, or more complexly, can include the sum of individual H-scores for each intensity level seen. By one method, the percentage of cells at each staining intensity level is calculated, and finally, an H-score is assigned using the following formula: [1×(% cells 1+)+2×(% cells 2+)+3×(% cells 3+)]. The final score, ranging from 0 to 300, gives more relative weight to higher-intensity membrane staining in a given tissue sample. The sample can then be considered positive or negative on the basis of a specific discriminatory threshold. The scoring 1, 2 and 3 were defined for each antibody and the same parameters were used to quantitate each antibody staining. The same applies for fluorescence.

### Example 2: Example of a BCC Model

FIG. 1 shows an example of an inducible and conditional mouse model of BCC. The mouse is genetically modified to express an inducible cassette which includes a sequence encoding a shRNA targeting Ptch1 gene and a sequence encoding a shRNA targeting p53 tumor suppressor gene. The cassette also includes a gene encoding for GFP, which acts as a marker for shRNA expression. Upstream of the genes, the cassette includes a Lox-STOP-Lox site (i.e., LoxP site) before a tetracycline-response element (TRE) to prevent any potential leakiness of the TRE. The inducible cassette is inserted into the Col1a1 safe-harbor site. The TRE promoter is activated by the transcriptional activator rtTA2, the gene for which is inserted into the constitutively expressed gene locus, ROSA26. In the absence of any inducers, the simultaneous presence of the LoxP site and lack of activation of the TRE block shRNA expression. The LoxP site is removed in the presence of a Cre recombinase in the nucleus. The mouse is genetically engineered to expresses a gene encoding Cre recombinase fused with estrogen receptor (Cre-ER), which is under the control of the skin cell-specific promoter K14. Therefore, Cre-ER is only expressed in the skin of mice. In the presence of tamoxifen, tamoxifen binds Cre-ER and induces its translocation to the nucleus for LoxP removal. Furthermore, in the presence of tetracycline or doxycycline, rtTA2 is expressed and binds and activates the TRE promoter. The presence of both the tamoxifen and tetracycline/doxycycline therefore induces expression of shRNAs and GFP in the cassette. Due to the specific activation of Cre-ER in the skin, expression of shRNAs and GFP in the cassette only occurs in the skin of mice.

### Example 3: Characterization of the Inducible BCC Model

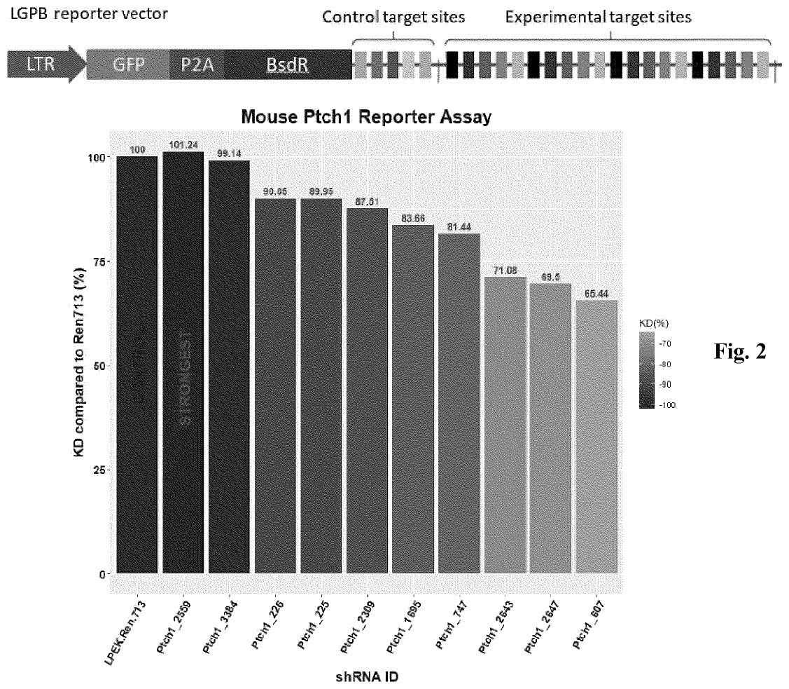

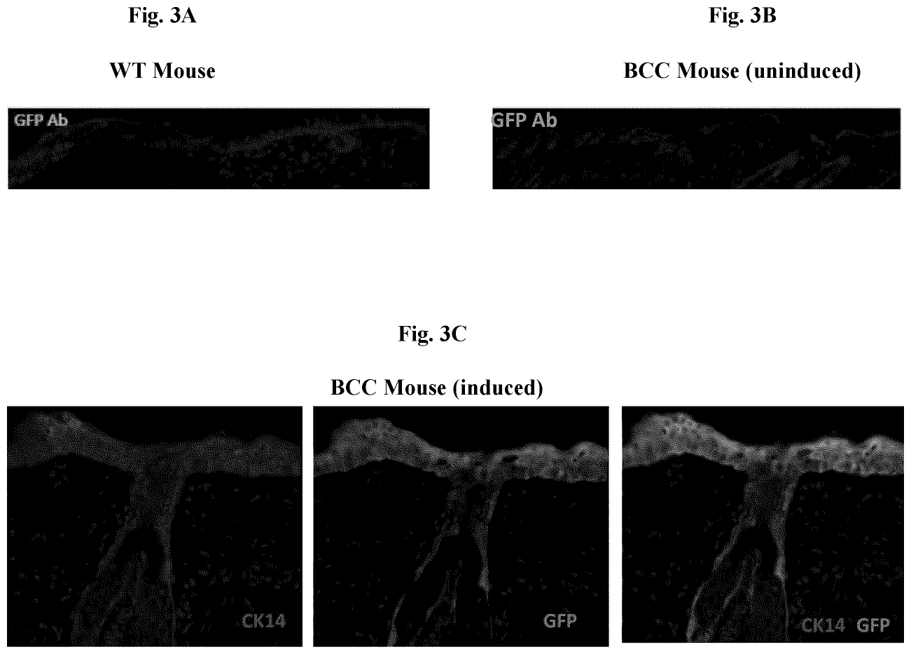

FIG. 2 shows the results of knockdown efficiency of a different shRNAs targeting mouse Ptch1 (shPtch1) in a reporter assay as described in Yuan et al. (2014). Knockdown efficiency was calculated by Ptch1 mRNA expression normalized to shRen713 (positive control), a reporter shRNA targeting Renilla Luciferase 713. The shRNA Ptch1.2559 (SEQ ID NO: 1) was shown to have the highest knockdown efficiency (101.24%) and was selected for the BCC model for the following experiments in FIGS. 3-12, along with the shRNA for the tumor suppressor gene p53, p53.1224 (SEQ ID NO: 11) as described in Premsirut et al. (2011).

Next, expression of shRNAs targeting Ptch1 and p53 was evaluated in the skins of induced BCC mice. FIG. 3A shows the representative result of immunofluorescence (IF) analysis of GFP in the skin of untreated WT mice or 9-weeks old BCC mice (i.e., BCC mice, 6 weeks-old mice following the induction). Positive GFP fluorescence is indicative of Ptch1 and p53 shRNA expression. No GFP fluorescence was apparent in untreated WT or BCC mice. FIG. 3B shows the representative result of IF of GFP and keratin 14 (CK14; basal skin) in BCC mice 14 weeks after induction with tamoxifen/doxycycline and UV-B radiation. FIG. 3C shows co-localization of GFP and CK14, suggesting elevated expression of Ptch1 and p53 shRNA expression in skin cells.

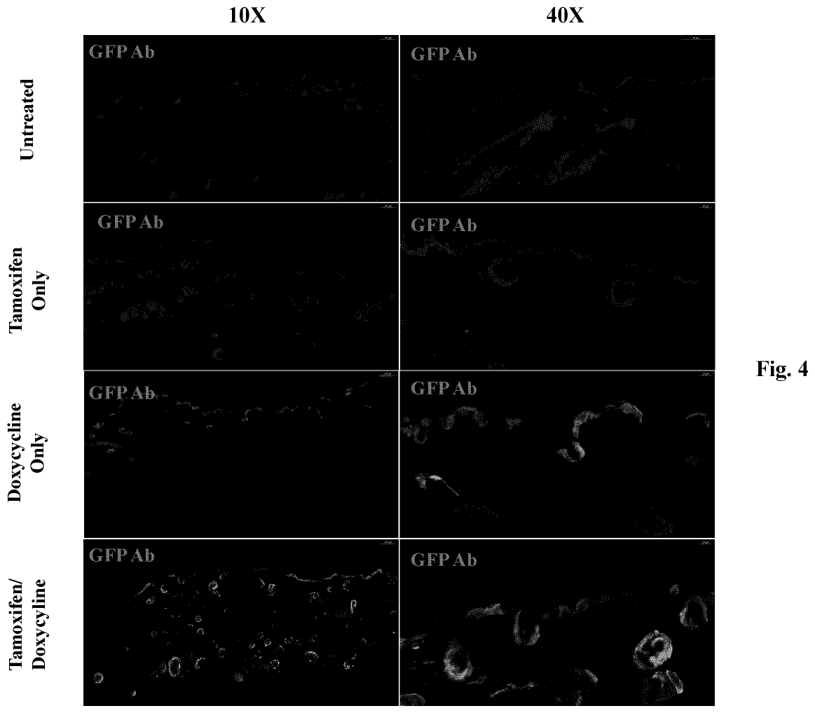

Next, shRNA expression was assessed in the skin of mice, either untreated, or after induction with either tamoxifen alone, doxycycline alone, or tamoxifen/doxycycline in the absence of UV. Fluorence microscopy of skin sections of mice showed no expression of GFP (i.e., shRNAs targeting Ptch1 and p53) when mice were either untreated or treated with tamoxifen alone. When treated with doxycycline only, low levels of GFP expression were observed (FIG. 4). Finally, when treated with tamoxifen and doxycycline, a significant increase in GFP expression was observed (FIG. 4). These data demonstrate that for efficient expression of GFP, treatment with tamoxifen and doxycycline was required. The GFP expression resulting from treatment with doxycycline alone was likely due to elevated levels of endogenous estrogen (likely from pregnancy) which bound to Cre-ER, allowing for its translocation to the nucleus and removal of LoxP.

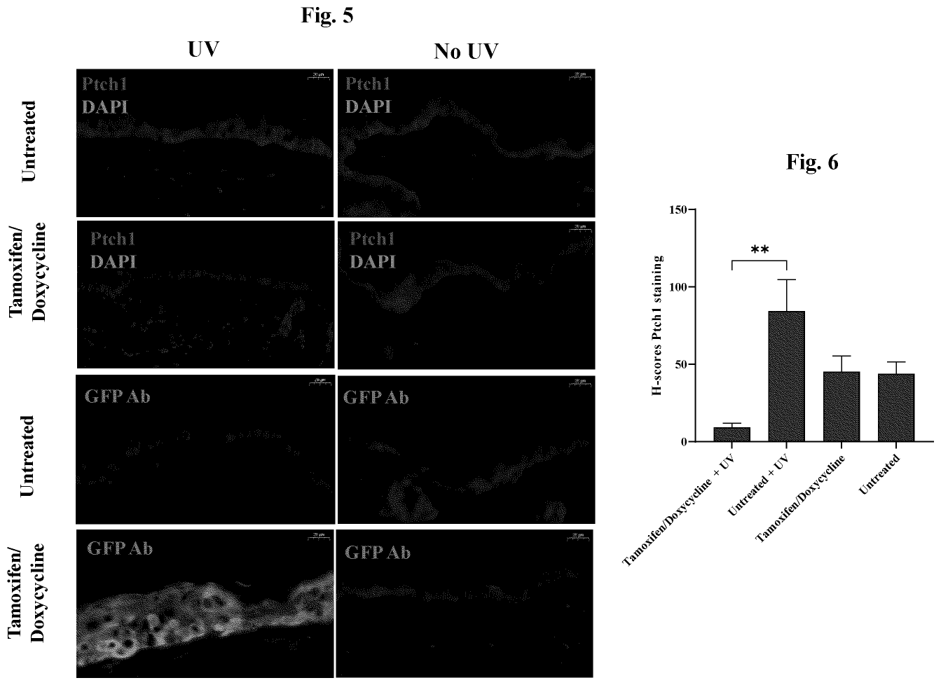

Next, Ptch1 knockdown in the skin of BCC mice was then evaluated. FIG. 5 shows the representative result of IF of Ptch1 in the skin of BCC mice, 14 weeks after induction with either tamoxifen/doxycycline, UV-B radiation, and/or untreated. DAPI levels indicate nuclear staining. FIG. 6 shows the quantitative expression of Ptch1 based on the IF results of FIG. 5. As shown, Ptch1 expression was significantly decreased in BCC mice induced with tamoxifen/doxycycline. Ptch1 knockdown was enhanced by exposure to UV, however, the effect of UV alone was not as significant.

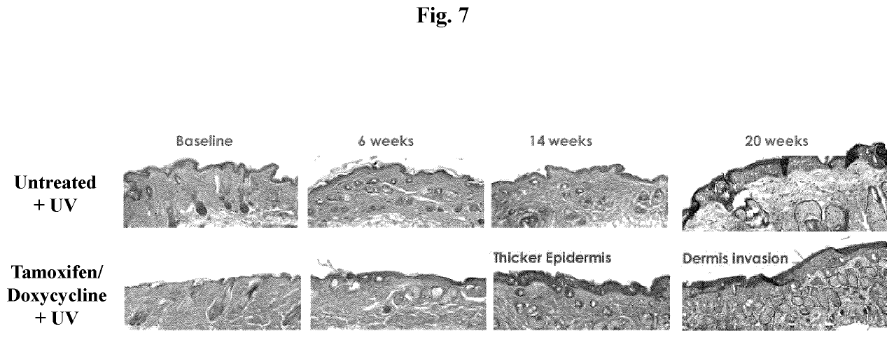

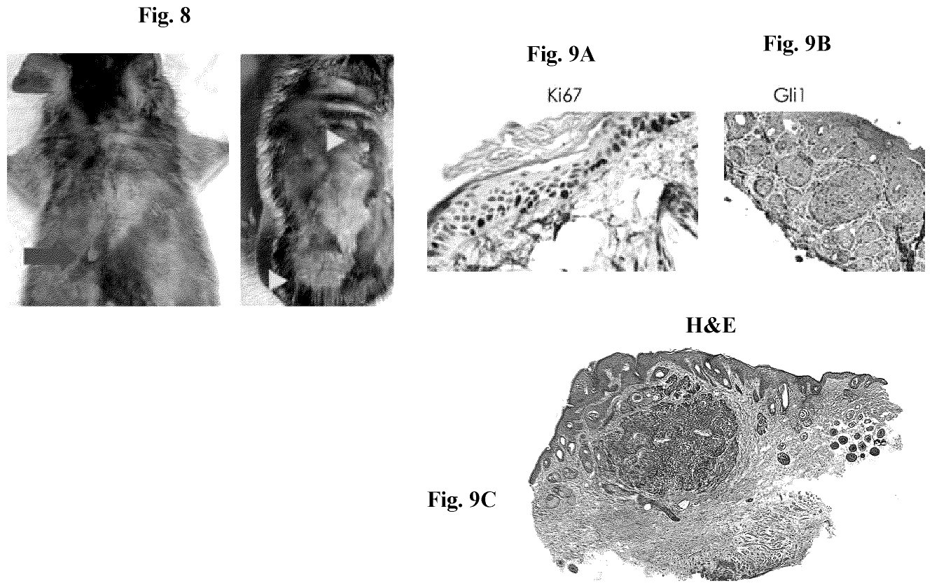

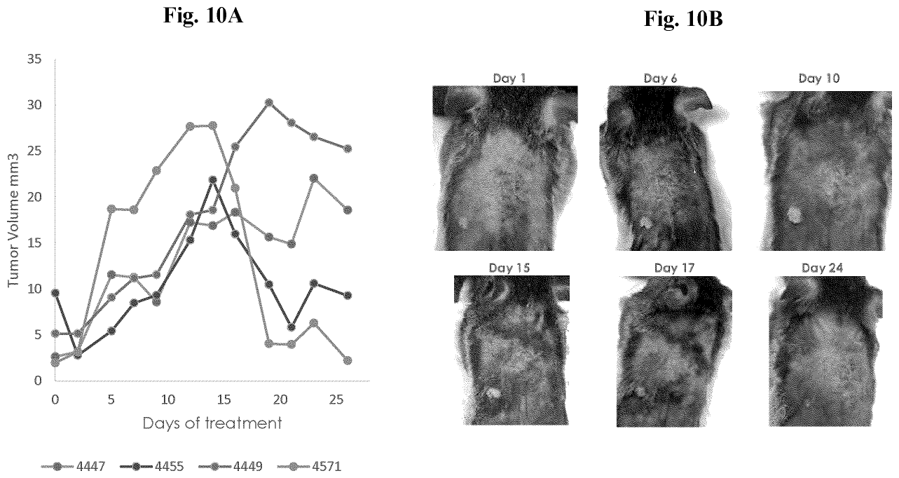

Next, the appearance and characterization of BCC tumors was evaluated in BCC mice. FIG. 7 shows the representative result of hematoxylin and eosin (H&E) staining of the skin of BCC mice upon induction with either tamoxifen/doxycycline or untreated in the presence of UV-B at baseline, 6 weeks, 14 weeks, or 20 weeks after induction. After 14 weeks after induction, thicker epidermis was observed in induced BCC mice, followed by apparent significant dermis invasion at 20 weeks. At 23 weeks post-induction, induced BCC mice developed visible BCC tumors (FIG. 8), which were characterized by elevated Ki67 and Gli1 staining by IHC and by BCC characteristic morphology using hematoxylin and eosin (H&E) staining (FIG. 9).

These data demonstrate a potent inducible animal model of BCC via a conditional knockdown of Ptch1 and p53 by shRNAs in the skin.

### Example 4: Use of the BCC Model to Identify and Determine the Efficiency of Anti-BCC Treatments

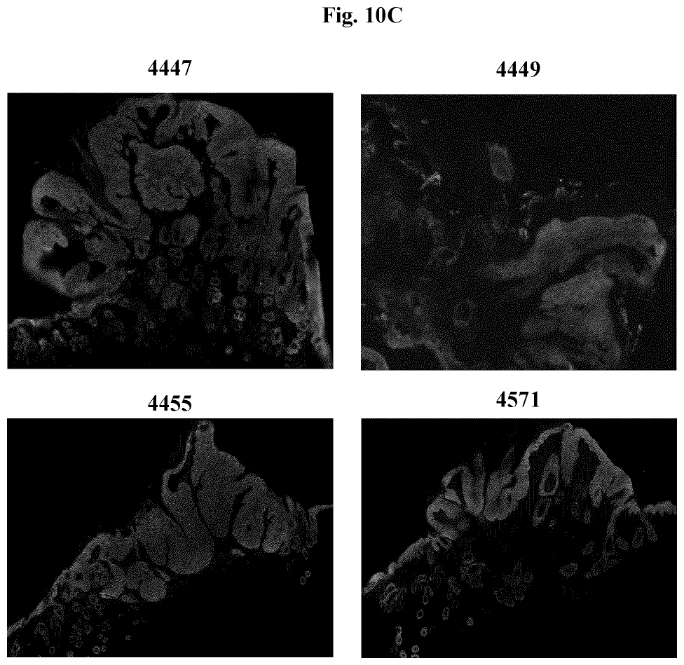

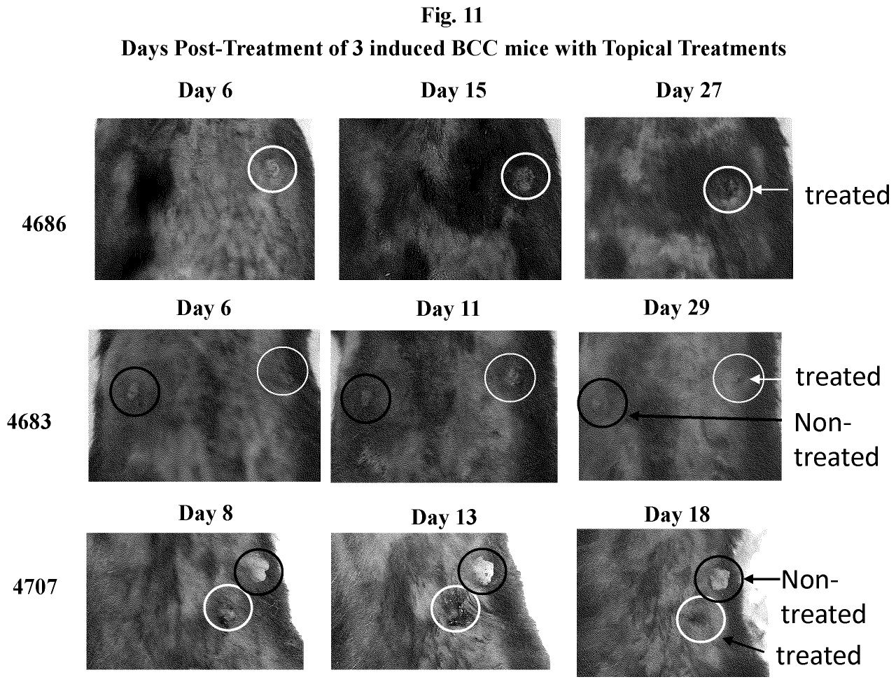

Next, the efficiency of a Gli-1-targeting topical formulation was evaluated in induced BCC mice. FIG. 10A shows the representative result of the effect of daily treatment with a topical formulation on the tumors of four BCC mice induced with tamoxifen/doxycycline and UV-B. Treatment began 22-26 weeks post-induction. A rapid decline in tumor volume was observed at least for mice 4455 and 4571 starting day 15 post-treatment. FIG. 10B shows images of the tumor at different time points of induced BCC mouse 4571 treated with the topical formulation. All mice exhibited elevated expression of GFP in the skin at 28 days after treatment, which is indicative of Ptch1 and p53 shRNA expression (FIG. 10C). In addition, three mice, 4686, 4683 and 4707 shown reduce tumor volumes after 4 weeks of daily topical treatment (FIG. 11). For 4707 and 4383, the treated tumors disappeared after 18 and 29 days of treatment and the non-treated tumors didn't change over the 4 weeks of treatment. For 4686, visible signs of scabbing and crusting was observed, which is indicative of healing and is typical seen in the treatment of human BCC lesions with vismodegib.

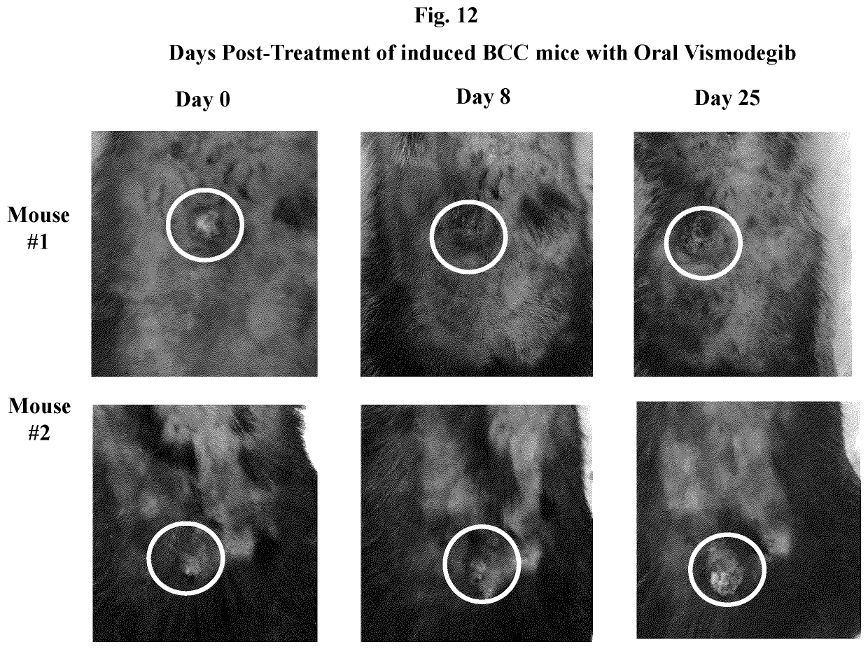

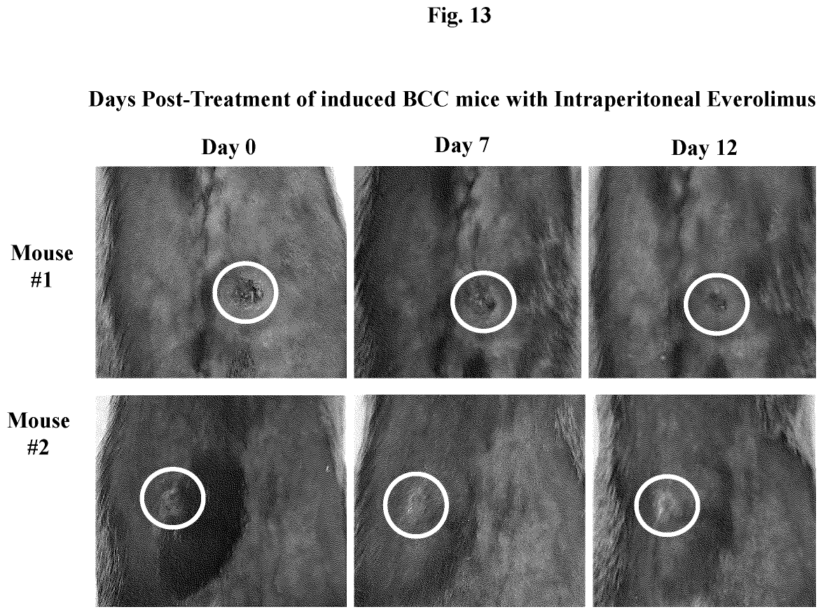

In addition to topical treatments, systemic treatments, such as via oral and intraperitoneal routes, were shown to be effective in treating BCC tumors in the BCC mouse model. BCC mice were treated with oral vismodegib, a cyclopamine derived-competitive antagonist of the smoothened receptor (SMO). After 18-25 days of treatment, visible signs of scabbing/crusting of the tumors were observed, as previously shown with the topical treatment (FIG. 12). Furthermore, FIG. 13 shows the representative result of the effect of treatment with intraperitoneal (i.p.) everolimus (3 mg/kg) in BCC mice induced with tamoxifen/doxycycline and UV-B. Everolimus, a derivative of rapamycin, is an mTOR inhibitor that is often used to treat BCC patients. Treatment began at 37-43 weeks post-induction. Representative images of the tumors (circle) at different time points of two different induced mice treated with i.p. everolimus are shown. After 7-12 days of treatment, visible signs of scabbing/crusting of the tumors were observed, as previously shown with the topical and oral treatments.

These data demonstrate the effectiveness of this induced BCC model to study a wide variety of anti-BCC treatments administered via multiple different routes, thereby increasing the potential of discovering novel treatments.

### REFERENCES

Nitzki F., et al., 2012. Patched Knockout Mouse Models of Basal Cell Carcinoma. Journal of Skin Cancer. Vol. 2012: 1-11.
Premsirut, P. K., et al., 2011. A Rapid and Scalable System for Studying Gene Function in Mice Using Conditional RNA Interference. Cell. 145: 145-158.
Yuan, T. L., et al., 2014. Development of siRNA payloads to target KRAS-mutant cancer. Cancer Discov. 4(10): 1182-1197.

1. A genetically-modified non-human animal whose genome comprises one or more sequences encoding a short hairpin RNA (shRNA) targeting Ptch1, and a shRNA targeting p53 or other tumor suppressor genes, wherein expression or co-expression of said shRNAs is inducible and in the skin of said non-human animal.

2. A genetically-modified non-human animal whose genome comprises:
an inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53;
and
a gene encoding a site-specific recombinase that cleaves the upstream cleavable sequence, wherein said gene is under the control of a skin cell-specific promoter and wherein expression of said gene is inducible.

3. The non-human animal of claim 2, wherein the cassette comprises an inducible promoter which controls the expression of the upstream cleavable sequence, first and second downstream sequences, wherein said inducible promoter is regulated by a transcriptional activator protein.

4. The non-human animal of claim 3, wherein the genome further comprises a gene encoding the transcriptional activator protein, wherein said gene is inserted into a constitutively expressed gene locus (e.g., ROSA26).

5. The non-human animal of claim 4, wherein expression of the gene encoding the transcriptional activator protein is inducible.

6. The non-human animal of any one of claims 3 to 5, wherein said inducible promoter is or comprises a tetracycline-response element (TRE) and wherein said transcriptional activator is a tetracycline transactivator (e.g., rtTA or rtTA2).

7. The non-human animal of any one of claims 3 to 6, wherein expression of the transcriptional activator is induced in the presence of tetracycline and/or doxycycline.

8. The non-human animal of any one of claims 2 to 7, wherein said skin cell-specific promoter is or comprises keratin 14 (K14), Krt6a, or K5.

9. The non-human animal of any one of claims 2 to 8, wherein said upstream cleavable sequence is or comprises a loxP site (i.e., Lox-STOP-Lox site).

10. The non-human animal of any one of claims 2 to 9, wherein said upstream cleavable sequence is or comprises a flippase recognition target (FRT) site.

11. The non-human animal of any one of claims 2 to 10, wherein said recombinase is or comprises a Cre recombinase or a flippase recombinase.

12. The non-human animal of any one of claims 2 to 11, wherein said recombinase is fused to a receptor, such as an estrogen receptor (e.g., ERT2).

13. The non-human animal of any one of claims 2 to 12, wherein activation of the recombinase is induced in the presence of tamoxifen.

14. The non-human animal of any one of claims 2 to 13, wherein said cassette further comprises a third downstream gene encoding a fluorescent marker or protein (e.g., green fluorescent protein [GFP]).

15. The non-human animal of any one of claims 2 to 14, wherein said genetic safe-harbour site is or comprises the Col1a1 gene locus.

16. The non-human animal of any one of claims 2 to 15, wherein the sequence encoding the short hairpin RNA (shRNA) targeting Ptch1 comprises the sequence of any one of SEQ ID NOs: 1-10.

17. The non-human animal of any one of claims 2 to 16, wherein the sequence encoding the short hairpin RNA (shRNA) targeting p53 comprises the sequence of SEQ ID NO: 11.

18. The non-human animal of any one of claims 2 to 17, wherein said non-human animal is a rodent (e.g., mouse, rat, hamster, or guinea pig), ferret, rabbit, pig, monkey, or primate.

19. The non-human animal of any one of claims 2 to 18, for use as a non-human animal model of inducible basal cell carcinoma (BCC).

20. A method of producing an inducible non-human animal, the method comprising:
inserting an inducible cassette into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53;
and
inserting into the genome of the non-human animal an inducible gene encoding a site-specific recombinase that cleaves the upstream cleavable sequence, wherein said gene is under the control of a skin cell-specific promoter.

21. The method of claim 20, wherein the cassette further comprises an inducible promoter which controls the expression of the upstream cleavable sequence, first and second downstream sequences, wherein said inducible promoter is regulated by a transcriptional activator protein.

22. The method of claim 21, further comprising inserting into the genome of the non-human animal a gene encoding the transcriptional activator protein, wherein said gene is inserted into a constitutively expressed gene locus (e.g., ROSA26).

23. The method of claim 22, wherein expression of the gene encoding the transcriptional activator protein is inducible.

24. The method of any one of claims 21 to 23, wherein said inducible promoter is or comprises a tetracycline-response element (TRE) and wherein said transcriptional activator is a tetracycline transactivator (e.g., rtTA or rtTA2).

25. The non-human animal of any one of claims 21 to 24, wherein expression of the transcriptional activator is induced in the presence of tetracycline and/or doxycycline.

26. The method of any one of claims 20 to 25, wherein said skin cell-specific promoter is or comprises keratin 14 (K14), Krt6a, or K5.

27. The method of any one of claims 20 to 26, wherein said upstream cleavable sequence is or comprises a loxP site (i.e., Lox-STOP-Lox site).

28. The method of any one of claims 20 to 27, wherein said upstream cleavable sequence is or comprises a flippase recognition target (FRT) site.

29. The method of any one of claims 20 to 28, wherein said recombinase is or comprises a Cre recombinase or a flippase recombinase.

30. The method of any one of claims 20 to 29, wherein said recombinase is fused to a receptor, such as an estrogen receptor (e.g., ERT2).

31. The method of any one of claims 20 to 30, wherein activation of the recombinase is induced in the presence of tamoxifen.

32. The method of any one of claims 20 to 31, wherein said cassette further comprises a third downstream gene encoding a fluorescent marker or protein (e.g., green fluorescent protein [GFP]).

33. The method of any one of claims 20 to 32, wherein said genetic safe-harbour site is or comprises the Col1a1 gene locus.

34. The method of any one of claims 20 to 33, wherein the sequence encoding the short hairpin RNA (shRNA) targeting Ptch1 comprises the sequence of any one of SEQ ID NOs: 1-10.

35. The method of any one of claims 20 to 34, wherein the sequence encoding the short hairpin RNA (shRNA) targeting p53 comprises the sequence of SEQ ID NO: 11.

36. The method of any one of claims 20 to 35, wherein said non-human animal is a rodent (e.g., mouse, rat, hamster, or guinea pig), ferret, rabbit, pig, monkey, or primate.

37. The method of any one of claims 20 to 36, wherein said non-human animal is the non-human animal as defined in any one of claims 1 to 19.

38. A method for inducing basal cell carcinoma (BCC) or BCC tumor(s) in a non-human animal, the method comprising:
providing a non-human animal whose genome comprises:
an inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53;
and
a gene encoding a site-specific recombinase that cleaves the upstream cleavable sequence,
wherein said gene is under the control of a skin cell-specific promoter and wherein expression of said gene is inducible;
and
administering a compound for inducing expression of the recombinase.

39. The method of claim 38, wherein the cassette further comprises an inducible promoter which controls the expression of the upstream cleavable sequence, first and second downstream sequences, wherein said inducible promoter is regulated by a transcriptional activator protein.

40. The method of claim 39, wherein the genome of the non-human animal further comprises a gene encoding the transcriptional activator protein, wherein said gene is inserted into a constitutively expressed gene locus (e.g., ROSA26).

41. The method of claim 40, wherein expression of the gene encoding the transcriptional activator protein is inducible.

42. The method of any one of claims 39 to 41, wherein said inducible promoter is or comprises a tetracycline-response element (TRE) and wherein said transcriptional activator is a tetracycline transactivator (e.g., rtTA or rtTA2).

43. The method of any one of claims 39 to 42, further comprising administering a compound for inducing expression of the transcriptional activator protein.

44. The method of claim 43, wherein the compound for inducing expression of the transcriptional regulator protein is or comprises tetracycline or doxycycline.

45. The method of any one of claims 38 to 44, wherein the compound for inducing activation of the recombinase is or comprises tamoxifen.

46. The method of any one of claims 38 to 45, further comprising exposing the non-human animal to ultraviolet radiation (e.g., UV-B) and/or ionizing radiation.

47. The method of any one of claims 38 to 46, wherein the non-human animal is produced by the method of any one of claims 20 to 37.

48. The method of any one of claims 38 to 47, wherein the non-human animal is the non-human animal as defined in any one of claims 1 to 19.

49. An isolated cell from the non-human animal as defined in any one of claims 1 to 19 or from a non-human animal produced by the method of any one of claims 20 to 37.

50. A cell comprising:
an inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53;
and
a gene encoding a site-specific recombinase that cleaves the upstream cleavable sequence, wherein said gene is under the control of a skin cell-specific promoter and wherein expression of said gene is inducible.

51. The cell of claim 50, wherein the cassette comprises an inducible promoter which controls the expression of the upstream cleavable sequence, first and second downstream sequences, wherein said inducible promoter is regulated by a transcriptional activator protein.

52. The cell of claim 51, further comprising a gene encoding the transcriptional activator protein, wherein said gene is inserted into a constitutively expressed gene locus (e.g., ROSA26).

53. The cell of claim 52, wherein expression of the gene encoding the transcriptional activator protein is inducible.

54. The cell of any one of claims 51 to 53, wherein said inducible promoter is or comprises a tetracycline-response element (TRE) and wherein said transcriptional activator is a tetracycline transactivator (e.g., rtTA or rtTA2).

55. The cell of any one of claims 51 to 54, wherein expression of the transcriptional activator is induced in the presence of tetracycline and/or doxycycline.

56. The cell of any one of claims 50 to 55, wherein said skin cell-specific promoter is or comprises keratin 14 (K14), Krt6a, or K5.

57. The cell of any one of claims 50 to 56, wherein said upstream cleavable sequence is or comprises a loxP site (i.e., Lox-STOP-Lox site).

58. The cell of any one of claims 50 to 57, wherein said upstream cleavable sequence is or comprises a flippase recognition target (FRT) site.

59. The cell of any one of claims 50 to 58, wherein said recombinase is or comprises a Cre recombinase or a flippase recombinase.

60. The cell of any one of claims 50 to 59, wherein said recombinase is fused to a receptor, such as an estrogen receptor (e.g., ERT2).

61. The cell of any one of claims 50 to 60, wherein activation of the recombinase is induced in the presence of tamoxifen.

62. The cell of any one of claims 50 to 61, wherein said cassette further comprises a third downstream gene encoding a fluorescent marker or protein (e.g., green fluorescent protein [GFP]).

63. The cell of any one of claims 50 to 62, wherein said genetic safe-harbour site is or comprises the Col1a1 gene locus.

64. The cell of any one of claims 50 to 63, wherein the sequence encoding the short hairpin RNA (shRNA) targeting Ptch1 comprises the sequence of any one of SEQ ID NOs: 1-10.

65. The cell of any one of claims 50 to 64, wherein the sequence encoding the short hairpin RNA (shRNA) targeting p53 comprises the sequence of SEQ ID NO: 11.

66. The cell of any one of claims 50 to 65, wherein said cell is a mammalian cell.

67. The cell of any one of claims 50 to 66, wherein said cell is from a non-human animal, such as a rodent (e.g., mouse, rat, hamster, or guinea pig), ferret, rabbit, pig, monkey, or primate.

68. The cell of any one of claims 50 to 67, wherein said cell is a skin cell, skin basal cell, germ cell, stem cell, embryonic stem cell, multipotent stem cell, pluripotent stem cell, totipotent stem cells, or a fertilized cell (e.g., oocyte, zygote).

69. A method of evaluating in a non-human animal, a candidate compound or intervention useful in the treatment, reduction or prevention of basal cell carcinoma (BCC) or BCC tumors, the method comprising the steps of:
providing the non-human animal as defined in any one of claims 1 to 19 or a non-human animal produced by the method of any one of claims 20 to 37;
inducing BCC or BCC tumor(s) by the method of any one of claims 38 to 48; and
administering the candidate compound or intervention to the non-human animal.

70. Use of the non-human animal as defined in any one of claims 1 to 19 for evaluating a candidate compound or intervention useful in the treatment, reduction or prevention of basal cell carcinoma (BCC) or BCC tumors.

71. A gene cassette or vector (e.g., plasmid) comprising:
an upstream cleavable sequence that blocks expression of downstream sequences or genes;
a first downstream sequence encoding a short hairpin RNA (shRNA) targeting Ptch1; and
a second downstream sequence encoding a shRNA targeting p53.

72. The gene cassette or plasmid of claim 71, wherein the expression of the cassette or vector is inducible.

73. A nucleotide sequence comprising:
a sequence encoding inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53;
and
a sequence encoding a gene encoding a site-specific recombinase that cleaves the upstream cleavable sequence, wherein said gene is under the control of a skin cell-specific promoter and wherein expression of said gene is inducible.

74. The nucleotide sequence of claim 73, wherein the cassette comprises a sequence encoding an inducible promoter which controls the expression of the upstream cleavable sequence, first and second downstream sequences, wherein said inducible promoter is regulated by a transcriptional activator protein.

75. The nucleotide sequence of claim 74, wherein the nucleotide sequence further comprises a sequence encoding a gene encoding the transcriptional activator protein, wherein said gene is inserted into a constitutively expressed gene locus (e.g., ROSA26).

76. The nucleotide sequence of claim 75, wherein expression of the gene encoding the transcriptional activator protein is inducible.

77. The nucleotide sequence of any one of claims 74 to 76, wherein said inducible promoter is or comprises a tetracycline-response element (TRE) and wherein said transcriptional activator is a tetracycline transactivator (e.g., rtTA or rtTA2).

78. The nucleotide sequence of any one of claims 74 to 77, wherein expression of the transcriptional activator is induced in the presence of tetracycline and/or doxycycline.

79. The nucleotide sequence of any one of claims 73 to 78, wherein said skin cell-specific promoter is or comprises keratin 14 (K14), Krt6a, or K5.

80. The nucleotide sequence of any one of claims 73 to 79, wherein said upstream cleavable sequence is or comprises a loxP site (i.e., Lox-STOP-Lox site).

81. The nucleotide sequence of any one of claims 73 to 80, wherein said upstream cleavable sequence is or comprises a flippase recognition target (FRT) site.

82. The nucleotide sequence of any one of claims 73 to 81, wherein said recombinase is or comprises a Cre recombinase or a flippase recombinase.

83. The nucleotide sequence of any one of claims 73 to 82, wherein said recombinase is fused to a receptor, such as an estrogen receptor (e.g., ERT2).

84. The nucleotide sequence of any one of claims 73 to 83, wherein activation of the recombinase is induced in the presence of tamoxifen.

85. The nucleotide sequence of any one of claims 73 to 84, wherein said cassette further comprises a third downstream sequence encoding a gene encoding a fluorescent marker or protein (e.g., green fluorescent protein [GFP]).

86. The nucleotide sequence of any one of claims 73 to 85, wherein said genetic safe-harbour site is or comprises the Col1a1 gene locus.

87. The nucleotide sequence of any one of claims 73 to 86, wherein the sequence encoding the short hairpin RNA (shRNA) targeting Ptch1 comprises the sequence of any one of SEQ ID NOs: 1-10.

88. The nucleotide sequence of any one of claims 73 to 87, wherein the sequence encoding the short hairpin RNA (shRNA) targeting p53 comprises the sequence of SEQ ID NO: 11.

89. A short hairpin RNA targeting Ptch1, said shRNA being encoded by a sequence having at least 50, 60, 70, 80, 90, 95, or 99% identity to the sequence of any one of SEQ ID NOs: 1-10.

90. A short hairpin RNA targeting Ptch1, said shRNA being encoded by the sequence of any one of SEQ ID NOs: 1-10.

91. A short hairpin RNA targeting Ptch1, said shRNA targeting a portion or region on SEQ ID NO: 12 that is at least 50, 60, 70, 80, 90, 95, 99%, or 100% identical to one or more of:
(xi) Nucleobases 2776-2796 of SEQ ID NO: 12;
(xii) Nucleobases 3601-3621 of SEQ ID NO: 12;
(xiii) Nucleobases 442-462 of SEQ ID NO: 12;
(xiv) Nucleobases 443-463 of SEQ ID NO: 12;
(xv) Nucleobases 2526-2546 of SEQ ID NO: 12;
(xvi) Nucleobases 1912-1933 of SEQ ID NO: 12;
(xvii) Nucleobases 964-986 of SEQ ID NO: 12;
(xviii) Nucleobases 2859-2880 of SEQ ID NO: 12;
(xix) Nucleobases 2864-2884 of SEQ ID NO: 12; and
(xx) Nucleobases 824-844 of SEQ ID NO: 12.

92. A composition or kit comprising one or more of the shRNAs as defined in any one of claims 89 to 91.

93. The shRNA of any one of claims 89-91 or the composition or kit of claim 92 for use in inducing BCC in a non-human animal.

94. A non-human animal expressing the gene cassette or vector of claim 71 or 72.

95. A cell comprising or expressing the gene cassette or vector of claim 71 or 72.

96. A genetically-modified non-human animal whose genome comprises:
an inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53.

97. The genetically modified non-human animal of claim 96, wherein the inducible cassette is heterzogyously expressed.

98. The genetically modified non-human animal of claim 97, wherein the inducible cassette is homozygously expressed.

99. A cell comprising:
an inducible cassette inserted into a genetic safe harbour site, wherein said cassette comprises:
an upstream cleavable sequence that blocks expression of downstream sequences or genes;
a first downstream sequence encoding a short hairpin RNA (shRNA) targeting Ptch1; and
a second downstream sequence encoding a shRNA targeting p53.


SEQUENCE LISTING (12 sequences)

SEQ ID NO: 1 - DNA, 61 bp, synthetic construct
  source 1..61: mol_type = other DNA
tcatgccaaacaattataaaatagtgaagccacagatgtattttataattgtttggcatga

SEQ ID NO: 2 - DNA, 61 bp, synthetic construct
  source 1..61: mol_type = other DNA
ccgaatttgatttcattgtcatagtgaagccacagatgtatgacaatgaaatcaaattcgg

SEQ ID NO: 3 - DNA, 61 bp, synthetic construct
  source 1..61: mol_type = other DNA
gggttgttacattcaaaagaatagtgaagccacagatgtattcttttgaatgtaacaaccc

SEQ ID NO: 4 - DNA, 61 bp, synthetic construct
  source 1..61: mol_type = other DNA
tgggttgttacattcaaaagatagtgaagccacagatgtatcttttgaatgtaacaaccca

SEQ ID NO: 5 - DNA, 61 bp, synthetic construct
  source 1..61: mol_type = other DNA
accagagaatatgacttcatatagtgaagccacagatgtatatgaagtcatattctctggt

SEQ ID NO: 6 - DNA, 61 bp, synthetic construct
  source 1..61: mol_type = other DNA
tggtggtattcaattttgctatagtgaagccacagatgtatagcaaaattgaataccacca

SEQ ID NO: 7 - DNA, 61 bp, synthetic construct
  source 1..61: mol_type = other DNA
tcctagaagagttaaagaaaatagtgaagccacagatgtattttctttaactcttctagga

SEQ ID NO: 8 - DNA, 61 bp, synthetic construct
  source 1..61: mol_type = other DNA
ccatcgacattagtcagttgatagtgaagccacagatgtatcaactgactaatgtcgatgg

SEQ ID NO: 9 - DNA, 61 bp, synthetic construct
  source 1..61: mol_type = other DNA
cgacattagtcagttgactaatagtgaagccacagatgtattagtcaactgactaatgtcg

SEQ ID NO: 10 - DNA, 61 bp, synthetic construct
  source 1..61: mol_type = other DNA
tcagataatagaatacctttatagtgaagccacagatgtataaaggtattctattatctga

SEQ ID NO: 11 - DNA, 61 bp, synthetic construct
  source 1..61: mol_type = other DNA
ccactacaagtacatgtgtaatagtgaagccacagatgtattacacatgtacttgtagtgg

SEQ ID NO: 12 - DNA, 7323 bp, Mus musculus
  source 1..7323: mol_type = genomic DNA
gcggccgcaaagacctcgggactcacgcgcaatgtggcaatggaaggcgcagggtctgagtccccggcagcggccacggccgcagcacccgcagcgcccgccgtgtgagcggcagcagcgggtctgtcacccggagccggagtccccggcggccagcagcgtcctcgcgagccgagcgcccaggcgcgcccggagcccgcggcggcggcggcaacatggcctcggctggtaacgccgccggggccctgggcaggcaggccggcggcgggaggcgcagacggaccgggggaccgcaccgcgccgcgccggaccgggactatctgcaccggcccagctactgcgacgccgccttcgctctggagcagatttccaaggggaaggctactggccggaaagcgccgctgtggctgagagcgaagtttcagagactcttatttaaactgggttgttacattcaaaagaactgcggcaagtttttggttgtgggtctcctcatatttggggccttcgctgtgggattaaaggcagctaatctcgagaccaacgtggaggagctgtgggtggaagttggtggacgagtgagtcgagaattaaattatacccgtcagaagataggagaagaggctatgtttaatcctcaactcatgatacagactccaaaagaagaaggcgctaatgttctgaccacagaggctctcctgcaacacctggactcagcactccaggccagtcgtgtgcacgtctacatgtataacaggcaatggaagttggaacatttgtgctacaaatcaggggaacttatcacggagacaggttacatggatcagataatagaatacctttacccttgcttaatcattacacctttggactgcttctgggaaggggcaaagctacagtccgggacagcatacctcctaggtaagcctcctttacggtggacaaactttgaccccttggaattcctagaagagttaaagaaaataaactaccaagtggacagctgggaggaaatgctgaataaagccgaagttggccatgggtacatggaccggccttgcctcaacccagccgacccagattgccctgccacagcccctaacaaaaattcaaccaaacctcttgatgtggcccttgttttgaatggtggatgtcaaggtttatccaggaagtatatgcattggcaggaggagttgattgtgggtggtaccgtcaagaatgccactggaaaacttgtcagcgctcacgccctgcaaaccatgttccagttaatgactcccaagcaaatgtatgaacacttcaggggctacgactatgtctctcacatcaactggaatgaagacagggcagccgccatcctggaggcctggcagaggacttacgtggaggtggttcatcaaagtgtcgccccaaactccactcaaaaggtgcttcccttcacaaccacgaccctggacgacatcctaaaatccttctctgatgtcagtgtcatccgagtggccagcggctacctactgatgcttgcctatgcctgtttaaccatgctgcgctgggactgctccaagtcccagggtgccgtggggctggctggcgtcctgttggttgcgctgtcagtggctgcaggattgggcctctgctccttgattggcatttcttttaatgctgcgacaactcaggttttgccgtttcttgctcttggtgttggtgtggatgatgtcttcctcctggcccatgcattcagtgaaacaggacagaataagaggattccatttgaggacaggactggggagtgcctcaagcgcaccggagccagcgtggccctcacctccatcagcaatgtcaccgccttcttcatggccgcattgatccctatccctgccctgcgagcgttctccctccaggctgctgtggtggtggtattcaattttgctatggttctgctcatttttcctgcaattctcagcatggatttatacagacgtgaggacagaagattggatattttctgctgtttcacaagcccctgtgtcagcagggtgattcaagttgagccacaggcctacacagagcctcacagtaacacccggtacagccccccacccccatacaccagccacagcttcgcccacgaaacccatatcactatgcagtccaccgttcagctccgcacagagtatgaccctcacacgcacgtgtactacaccaccgccgagccacgctctgagatctctgtacagcctgttaccgtcacccaggacaacctcagctgtcagagtcccgagagcaccagctctaccagggacctgctctcccagttctcagactccagcctccactgcctcgagcccccctgcaccaagtggacactctcttcgtttgcagagaagcactatgctcctttcctcctgaaacccaaagccaaggttgtggtaatccttcttttcctgggcttgctgggggtcagcctttatgggaccacccgagtgagagacgggctggacctcacggacattgttccccgggaaaccagagaatatgacttcatagctgcccagttcaagtacttctctttctacaacatgtatatagtcacccagaaagcagactacccgaatatccagcacctactttacgaccttcataagagtttcagcaatgtgaagtatgtcatgctggaggagaacaagcaacttccccaaatgtggctgcactactttagagactggcttcaaggacttcaggatgcatttgacagtgactgggaaactgggaggatcatgccaaacaattataaaaatggatcagatgacggggtcctcgcttacaaactcctggtgcagactggcagccgagacaagcccatcgacattagtcagttgactaaacagcgtctggtagacgcagatggcatcattaatccgagcgctttctacatctacctgaccgcttgggtcagcaacgaccctgtagcttacgctgcctcccaggccaacatccggcctcaccggccggagtgggtccatgacaaagccgactacatgccagagaccaggctgagaatcccagcagcagagcccatcgagtacgctcagttccctttctacctcaacggcctacgagacacctcagactttgtggaagccatagaaaaagtgagagtcatctgtaacaactatacgagcctgggactgtccagctaccccaatggctaccccttcctgttctgggagcaatacatcagcctgcgccactggctgctgctatccatcagcgtggtgctggcctgcacgtttctagtgtgcgcagtcttcctcctgaacccctggacggccgggatcattgtcatggtcctggctctgatgaccgttgagctctttggcatgatgggcctcattgggatcaagctgagtgctgtgcctgtggtcatcctgattgcatctgttggcatcggagtggagttcaccgtccacgtggctttggcctttctgacagccattggggacaagaaccacagggctatgctcgctctggagcacatgtttgctcccgttctggacggtgctgtgtccactctgctgggtgtactgatgcttgcagggtccgaatttgatttcattgtcagatacttctttgccgtcctggccattctcaccgtcttgggggttctcaatggactggttctgctgcctgtcctcttatccttctttggaccgtgtcctgaggtgtctccagccaatggcctaaaccgactgcccactccttcgcctgagccgcctccaagtgtcgtccggtttgccgtgcctcctggtcacacgaacaatgggtctgattcctccgactcggagtacagctctcagaccacggtgtctggcatcagtgaggagctcaggcaatacgaagcacagcagggtgccggaggccctgcccaccaagtgattgtggaagccacagaaaaccctgtctttgcccggtccactgtggtccatccggactccagacatcagcctcccttgacccctcggcaacagccccacctggactctggctccttgtcccctggacggcaaggccagcagcctcgaagggatccccctagagaaggcttgcggccacccccctacagaccgcgcagagacgcttttgaaatttctactgaagggcattctggccctagcaatagggaccgctcagggccccgtggggcccgttctcacaaccctcggaacccaacgtccaccgccatgggcagctctgtgcccagctactgccagcccatcaccactgtgacggcttctgcttcggtgactgttgctgtgcatcccccgcctggacctgggcgcaacccccgaggggggccctgtccaggctatgagagctaccctgagactgatcacggggtatttgaggatcctcatgtgccttttcatgtcaggtgtgagaggagggactcaaaggtggaggtcatagagctacaggacgtggaatgtgaggagaggccgtgggggagcagctccaactgagggtaattaaaatctgaagcaaagaggccaaagattggaaagccccgcccccacctctttccagaactgcttgaagagaactgcttggaattatgggaaggcagttcattgttactgtaactgattgtattattttgtgaaatatttctataaatatttaaaaggtgtacacatgtaatatacatggaaatgctgtacagtctatttcctggggcctctccactcctgccccagagtggggagaccacaggggccctttcccctgtgtacattggtctctgtgccacaaccaagcttaacttagttttaaaaaaaatctcccagcatatgtcgctgctgcttaaatattgtataatttacttgtataattctatgcaaatattgcttatgtaataggattatttgtaaaggtttctgtttaaaatattttaaatttgcatatcacaaccctgtggtaggatgaattgttactgttaacttttgaacacgctatgcgtggtaattgtttaacgagcagacatgaagaaaacaggttaatcccagtggcttctctaggggtagttgtatatggttcgcatgggtggatgtgtgtgtgcatgtgactttccaatgtactgtattgtggtttgttgttgttgttgctgttgttgttcatttttgtgttttttgttgctttgtatgatcttagctctggcctaggtgggctgggaaggtccaggtctttttctgtcgtgatgctggtggaaaggtgaccccaatcatctgtcctattctctgggactattcaagagaagccagatttgcttcatgcctgtgtgtggcagcttctgaggtcatgggtagccctccaagcacctctgcttgggtttcaaagagaagatgttctcacaaacattgtgctgcctattagggcctctatatagtcagcagtcagcactagtggtgaaagattggacaatgttgcctgatggtgagactcagccgggtcatgagattaccctctaggggtgtgtggttgttcctttctgatgatcacacatacacacagcctcccacccccatactccagatacaggcccaaaaaaacctatttcactgaaggtctatattcgagcctttacaaatgatagtccctcccttgctatgactgcctggttggagcaggctaggctaatcgtggttcagtcaactgtgtggggagagtttctaaggactcttttccacacggtcttttcctgtgtatactttcttctcttccccactgttagcatccatccagtgtgatgtctgtgaaagcaggaagatgccacctcgtaaaagcatatagggtaagctctttgaaccaggtgttgggctttagagtttgattgtgttctccttccccaaggtttgggtttctttgtctctggtcagcaactagtagatagggaaggaaatgcccttattccttgcaagtgaacattgaggacttgtctcagaggaaggaagcctagtgctcatggaagctgttgtgaggaatgtactctgtctgcatgcagcttccctgaatccctcctatccttcatggaaaaatttaaggagatgggtccctagtagtaccaataatctagtgccctcagatcctaaaatttatcccccaaattgtaaggtactaattggaagccacctgtgactaccgtgtgtgtgtgtgtctgtgtctgtctgtatatccgtgtgcgcgtgcgcgccagaagccctggccatccactgctactgtcttaatgcctgtcaagccactgtctgcctacccaacaccagctctgctcggatatcttgcaccctgagttgaggagggagatgtagagtgcggaagccaccttggctgtggttcttgattgtgtccctcatgcctgagccttgtgcatgtggcagaaggaagtttgtacagcctctcggcttctgtgcatcatcatgagtcccatcagccaaggcaggaaggacacagagctggcaggagactaaagtcagagagtgtgtgtctctgtctgtctctctcatagttttattttgtctgtattgtttgttcatttggatgttttaatttgtaaaagaaaagatctttgctgatatttataattttgtatcataagaatgtcctccagaatttgtcatgccagtttataacaagaagaaaaaattgcagggattttatttctattggaaacacttattgcagttatgttttacttttgaacagaagttttatttgtatagagtgcttactaatgttaaatagttcagagtatataacatttacattaaggactcatggtaggttttagtgtaaggagtttaaaggaaataaatattcaaactgggtctcgtctgccaatttgggtggaaatgagtttgtgtcacttcaattacaaagatgaaagtatgccatataatttatttatatgaaaatttatttttgtagtgtacatagtagtcatcaagtctttcgacagaagtatatttttaaagaatttatatgtgatgaaatccataatgtctggaactttgctgagacatgggtgtgaggacacgtttcgttataaatgacagcaagggagagaagagagtatgttttaacagtgttaggagagtacacgtgagcagtgatccatgtgattggaaagtatcggtgtgaacatggtgacctagtgcggttctcagatgaaaatgtacaaaactctctaaatattaatgttcaaacactgatagaaattctaacatgaataaagataatataacttgttggtttaaaaaaaaaaaaaaaaaa